(19) United States
(12) Patent Application Publication
(10) Pub. No.: US 2013/0243794 A1
(43) Pub. Date: Sep. 19, 2013

(54) METHODS FOR PREDICTING AND TREATING INFECTION-INDUCED ILLNESSES AND PREDICTING THE SEVERITY OF INFECTION-INDUCED ILLNESSES

(71) Applicant: Carl J. Hauser, Boston, MA (US)

(72) Inventor: Carl J. Hauser, Boston, MA (US)

(73) Assignee: Beth Israel Deaconess Medical Center, Inc., Boston, MA (US)

(21) Appl. No.: 13/990,605

(22) PCT Filed: Dec. 5, 2011
(86) PCT No.: PCT/US11/63335

§ 371 Date: May 30, 2013

Related U.S. Application Data
(60) Provisional application No. 61/419,502, filed on Dec. 3, 2010.

(51) Int. Cl. C12Q 1/68; A61K 31/4706; A61K 38/48; A61K 38/17; A61K 39/395; A61K 45/06

(52) U.S. Cl. CPC C12Q 1/689; A61K 39/3955; A61K 45/06; A61K 38/4866; A61K 38/1709; A61K 31/4706
USPC 424/172.1; 435/6.12; 424/94.64; 514/20.5; 514/313

(57) ABSTRACT

The invention provides methods for determining the propensity to develop, or the presence of, one or more infection-induced illness(es). The invention further provides methods of treating an infection (e.g., bacterial infection), methods of diagnosing an infection-induced illness in a subject, and methods of predicting the future severity of one or more infection-induced illness(es). Also provided are kits for determining the likelihood or propensity of a subject to develop one or more infection-induced illness(es) and kits for diagnosing one or more infection-induced illness(es) in a subject and identifying a subject as having an increased risk of later developing one or more severe infection-induced illness(es).

### BACKGROUND OF THE INVENTION

The present invention relates to the clinical diagnosis of, or a prediction of the development of, infection-induced illnesses, the prediction of the severity of infection-induced illness(es), and methods of treating an infection (e.g., a bacterial infection).

Subjects with active infections (e.g., local or systemic bacterial infections) and subjects that previously experienced an infection may develop one or more infection-induced illness(es). Non-limiting examples of infection-induced illnesses include organ failure, hypotension, seizures, shock, increased heart rate, tachypnea, decreased arterial pressure of CO₂, and hemolytic-uremic syndrome. There are presently no diagnostic or predictive assays for determining whether a subject having a bacterial infection or a subject who has previously experienced an infection will later develop one or more infection-induced illness(es). There are also no diagnostic assays for predicting the severity of infection-induced illness(es). The ability to determine the propensity of a subject to develop an infection-induced illness or predict the severity of an infection-induced illness will allow medical professionals to cost-effectively triage and monitor subjects that have an increased propensity to later develop one or more infection-induced illness(es) or patients that are predicted to develop one or more severe infection-induced illness(es). The present invention provides methods for predicting and determining the propensity of a subject to develop one or more infection-induced illness(es) and methods for treating a subject having an infection (e.g., bacterial infection). The present invention also provides methods for predicting and/or determining that a subject does not have one or more infection-induced illness(es) and methods for treating a subject under such circumstances.

### SUMMARY OF THE INVENTION

In a first aspect, the invention provides methods for determining the likelihood that a subject will develop one or more (e.g., two, three, four, or five) infection-induced illness(es) and/or a method of predicting the severity of one or more infection-induced illness(es) in the future requiring the steps of: measuring the amount of microbial (e.g., bacterial, fungal, or viral) nucleic acid or peptide in a sample from the subject; measuring the amount of a mitochondrial nucleic acid or peptide in the sample; and determining whether the subject has an increased likelihood of later developing an infection-induced illness by comparing the amount of microbial nucleic acid or peptide measured with the amount of mitochondrial nucleic acid or peptide measured, wherein an increased ratio of the amount of mitochondrial nucleic acid or peptide to the amount of microbial nucleic acid or peptide indicates a subject with an increased likelihood of later developing one or more infection-induced illness(es) or indicates that the one or more infection-induced illness(es) may be severe in the future.

In a second aspect, the invention provides methods of identifying a subject with an increased propensity to develop one or more (e.g., two, three, four, or five) infection-induced illness(es) or an increased propensity to develop one or more severe infection-induced illness(es) in the future requiring the steps of: measuring the amount of microbial (e.g., bacterial, fungal, or viral) nucleic acid or peptide in a sample from the subject; measuring the amount of a mitochondrial nucleic acid or peptide in the sample; and comparing the amount of microbial nucleic acid or peptide measured with the amount of mitochondrial nucleic acid or peptide measured, wherein an increased ratio of the amount of mitochondrial nucleic acid or peptide to the amount of microbial nucleic acid or peptide identifies a subject that has an increased propensity to later develop one or more infection-induced illness(es) and/or an increased propensity to develop one or more severe infection-induced illness(es) in the future.

In a further aspect, the invention provides methods of diagnosing one or more (e.g., two, three, four, or five) infection-induced illness(es) or predicting the severity of one or more infection-induced illness(es) in a subject requiring the steps of: measuring the amount of microbial (e.g., bacterial, fungal, or viral) nucleic acid or peptide in a sample from the subject; measuring the amount of a mitochondrial nucleic acid or peptide in the sample; and comparing the amount of microbial nucleic acid or peptide measured with the amount of mitochondrial nucleic acid or peptide, wherein an increased ratio of the amount of mitochondrial nucleic acid or peptide to the amount of microbial nucleic acid or peptide indicates that the subject has one or more infection-induced illness(es) and/or indicates that the one or more infection-induced illness(es) may be severe in the future.

The invention also provides methods of treating a subject with a microbial (e.g., bacterial, fungal, or viral) infection comprising the steps of: measuring the amount of microbial (e.g., bacterial, fungal, or viral) nucleic acid or peptide in a sample from the subject; measuring the amount of a mitochondrial nucleic acid or peptide in the sample; comparing the amount of microbial nucleic acid or peptide measured with the amount of mitochondrial nucleic acid or peptide measured; and administering to the subject having an increased ratio of the amount of mitochondrial nucleic acid or peptide to the amount of microbial nucleic acid or peptide one or more anti-inflammatory agents and administering one or more antimicrobial agents (e.g., anti-FPR1 antibodies, cyclosporine H, activated protein C, chloroquine, and/or anti-TLR9 antibodies).

The invention also features a method for predicting and/or determining whether a subject (e.g., a mammal, such as a human) has one or more infection-induced illness(es). The method involves determining the amount of a mitochondrial nucleic acid or peptide in a sample from the subject (e.g., a blood sample) and/or determining the amount of microbial (e.g., bacterial, fungal, or viral) nucleic acid or peptide in the same or a different sample from the subject and from the determination(s) predicting and/or determining whether the subject has one or more infection-induced illness(es). In an embodiment, the method involves determining the amount of the mitochondrial nucleic acid or peptide in the sample and not determining the amount of microbial (e.g., bacterial, fungal, or viral) nucleic acid or peptide in the sample. In this embodiment, an increased amount of mitochondrial nucleic acid or peptide in the sample, relative to a control subject lacking tissue injury or microbial infection, indicates the subject may have tissue injury but not a microbial infection. In another embodiment, the method involves determining the amount of microbial (e.g., bacterial, fungal, or viral) nucleic acid or peptide in the sample and not determining the amount of mitochondrial nucleic acid or peptide in the sample. In this embodiment, an increased amount of microbial (e.g., bacterial, fungal, or viral) nucleic acid or peptide in the sample, relative to a control subject lacking tissue injury or microbial infection, indicates the subject may have a microbial infection but not tissue injury. In yet another embodiment, the method involves determining a ratio of the amount of mitochondrial nucleic acid or peptide in the sample and the amount of microbial (e.g., bacterial, fungal, or viral) nucleic acid or peptide in the sample. In this embodiment, a ratio of, e.g., about 10:1 to about 1000:1 (or one or more of the other ratios discussed below) indicates the subject may have a tissue injury but not a microbial infection or may be more susceptible to developing one or more infection-induced illnesses in the future.

In any of the treatment methods, the subject may be administered one or more (e.g., at least two, three, or four) doses of one or more (e.g., two, three, or four) antimicrobial agents prior to the administration of one or more (e.g., at least two, three, or four) doses of one or more (e.g., two, three, or four) anti-inflammatory agents. In additional embodiments of any of the treatment methods, the one or more anti-inflammatory agents is administered at least 12 hours (e.g., at least 24 hours, 2 days, 3 days, or 4 days) after the administration of the one or more antimicrobial agents. In any of the above methods of treatment, the treatment reduces (e.g., by at least 10%, 20%, 30%, 40%, 50%, 60%, 70%, 80%, 90%, or 95%) the likelihood of developing one or more (e.g., two, three, or four) infection-induced illness(es) or reduces (e.g., by at least 10%, 20%, 30%, 40%, 50%, 60%, 70%, 80%, 90%, or 95%) the likelihood of death resulting from one or more (e.g., two, three, or four) infection-induced illnesses. In additional embodiments of the above treatment methods, the subject has been previously diagnosed as having an infection (e.g., a bacterial, fungal, or viral infection).

The invention further provides kits for determining the likelihood that a subject will develop one or more infection-induced illness(es) or predicting the future severity of one or more infection-induced illness(es) in the future, identifying a subject with an increased propensity to later develop one or more infection-induced illness(es) or an increased propensity to later develop one or more severe infection-induced illness(es), diagnosing one or more infection-induced illness(es) in a subject, identifying a subject at increased risk for later developing one or more severe infection-induced illness(es) that contain: one or more first oligonucleotide primers effective for the amplification of a microbial (e.g., bacterial, fungal, or viral) nucleic acid; one or more second oligonucleotide primers effective for the amplification of a mitochondrial nucleic acid; and instructions for using the first and second oligonucleotide primers to determine the likelihood of a subject to later develop one or more infection-induced illness(es) or one or more severe infection-induced illness(es), identify a subject as having an increased propensity to later develop one or more infection-induced illness(es) or one or more severe infection-induced illness(es), predict the future severity of one or more infection-induced illnesses, diagnose a subject as having one or more infection-induced illness(es), or identify a subject that has an increased propensity to later develop a severe infection-induced illness(es).

The invention further provides kits for determining the likelihood that a subject will develop one or more infection-induced illness(es) or predicting the future severity of one or more infection-induced illness(es) in the future, identifying a subject with an increased propensity to later develop one or more infection-induced illness(es) or an increased propensity to later develop one or more severe infection-induced illness(es), diagnosing one or more infection-induced illness(es) in a subject, identifying a subject at increased risk for later developing one or more severe infection-induced illness(es) that contain: one or more antibodies that specifically bind to one or more microbial (e.g., bacterial, fungal, or viral) peptides; one or more antibodies that specifically bind to one or more mitochondrial peptides; and instructions for using the antibodies to determine the likelihood of a subject to later develop one or more infection-induced illness(es) or one or more severe infection-induced illness(es), identify a subject as having an increased propensity to later develop one or more infection-induced illness(es) or one or more severe infection-induced illness(es), predict the future severity of one or more infection-induced illnesses, diagnose a subject as having one or more infection-induced illness(es), or identify a subject that has an increased propensity to later develop a severe infection-induced illness(es).

In any of the above aspects, the one or more infection-induced illness(es) may be selected from the group of: organ failure, hypotension, seizures, shock, increased heart rate, tachypnea, decreased arterial pressure of CO₂, and hemolytic-uremic syndrome. In any of the above aspects, the one or more infection-induced illness(es) results in death. In any of the above aspects, the subject may have an infection (e.g., a bacterial infection, such as a local or systemic bacterial infection). In any of the above aspects, the subject may not demonstrate any symptoms of severe septic shock. In any of the above aspects, organ failure may be treated, for example, by endotracheal intubation, ventilator use, and/or renal dialysis.

In any of the above aspects, the bacterial infection may be caused by one or more bacteria selected from the group of: Bacillus athracis, Vibrio cholera, Bordetella pertussis, Eschericia coli, Clostridium tetani, Clostridium perfringes, Clostridium difficile, Clostridium botulinum, Listeria monocytogenes, Streptococcus spp., Staphylococcus aureus, Mycobacterium tuberculosis, Corynebacterium diphtheria, Shigella dysenteriae, Pseudomonas aeriginosa, and Bacillus thuringiensis. In additional embodiments, the bacterial infection may be caused by Bacillus athracis, Eschericia coli, or Staphylococcus aureus. In any of the above aspects, the sample may be obtained from a subject within 24 hours (e.g., within 20 hours, 16 hours, 12 hours, 8 hours, or 4 hours) of an initial presentation of the subject to a medical professional. In additional embodiments of the above methods, the sample may be obtained from a subject at least 24 hours after an initial presentation to a medical professional. In further embodiments of the above methods, the sample may be obtained from a subject already admitted to a medical facility.

In any of the above methods, a subject determined to have an increased likelihood or an increased propensity of later developing an infection-induced illness, or an increased likelihood of later developing one or more severe infection-induced illness(es) is admitted to a medical facility. In any of the above methods, a subject determined to have an increased likelihood or an increased propensity of later developing an infection-induced illness or a severe infection-induced illness is admitted to a medical facility for a longer period of time (a longer stay in a medical facility). For example, the assay may be performed on a subject already admitted to a medical facility and a medical professional determines that the subject should be admitted for a longer stay in the medical facility. In additional embodiments of the above methods, a subject identified (diagnosed) as having one or more infection-induced illness(es) or identified as being at risk of later developing a severe infection-induced illness is admitted to a medical facility.

In additional aspects of all the above methods, the subject may have been potentially exposed to an endotoxic bacterium or a composition containing an endotoxin (e.g., anthrax toxin or shiga toxin). In additional aspects of these methods, the sample may be obtained from the subject within 3 to 24 hours after a potential exposure to an endotoxic bacterium or a composition containing an endotoxin.

In any of the above aspects, the mitochondrial nucleic acid encodes cytochrome B, cytochrome C oxidase subunit III, or NADH dehydrogenase. In any of the above aspects, the mitochondrial nucleic acid encodes cytochrome B.

In any of the above aspects, the methods may also involve measuring or determining the amount of a control nucleic acid or peptide (e.g., a housekeeping gene or peptide, such as beta-actin or others known in the art) for the purpose of determining background levels (and, e.g., to reduce the number of false positives).

In yet other embodiments of all aspects of the invention, the method may be performed one or more times over a defined period of time. For example, one or more samples may be obtained from a subject within a 1 to 48 hour period (or more) and tested according to the methods described above. In particular, a sample may be obtained and tested every 30 minutes or every 1, 2, 3, 4, 5, 10, 12, 24, 36, or 48 hours or more. Changes in the amounts of mitochondrial and/or microbial nucleic acids and peptides over time can be used to assess whether a subject has, does not have, or is developing one or more infection-induced illnesses. The amounts of mitochondrial and/or microbial nucleic acids and peptides can also be monitored over time to assess whether a subject is responding to one or more therapies.

Probes and primers for amplifying mitochondrial and/or microbial nucleic acids other than those described herein may be used in the context of the present invention. Preferably, the invention features probes and primers capable of amplifying the target nucleic acids without the amplification of non-target nucleic acids.

By “anti-inflammatory agent” is meant an agent that reduces (e.g., by at least 10%, 15%, 20%, 25%, 30%, 35%, 40%, 45%, 50%, 55%, 60%, 65%, 70%, 75%, 80%, 85%, 90%, or 95%) one or more (e.g., two, three, four, or five) symptoms of inflammation when administered (e.g., orally, intravenously, intraarterially, and subcutaneously) to a subject. Non-limiting examples of anti-inflammatory agents include non-steroidal anti-inflammatory agents (e.g., ibuprofen, naproxen, fenoprofen, ketoprofen, flurbiprofen, oxaprozin, indomethacin, sulindac, etodolac, ketorolac, diclofenac, nabumetone, piroxicam, meloxicam, tenoxicam, droxicam, lornoxicam, isoxicam, mefenaic acid, meclofenamic acid, tolfenamic acid, celecoxib, rofecoxib, valdecoxib, parecoxib, lumiracoxib, etoricoxib, firoxocib, nimesulide, and licofelone), immunosuppressive agents (e.g., methotrexate, azathioprine, basiliximab, daclizumab, cyclosporine, tacrolimus, sirolimus, voclosporin, infliximab, etanercept, adalimumab, mycophenolic acid, fingolimod, pimecrolimus, thalidomide, lenalidomide, anakinra, deferolimus, everolimus, temsirolimus, zotarolimus, biolimus A9, and elsilimomab), corticosteroids (e.g., hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate), anti-FPR1 antibodies, cyclosporine H, activated protein C, chloroquine, and anti-TLR9 antibodies.

By “antimicrobial agent” is meant an agent that kills or inhibits (e.g., by at least 10%, 20%, 30%, 40%, 50%, 60%, 70%, 80%, 90%, or 100%) the growth of a microorganism (e.g., a bacterium, fungus, or protozoa) when administered to a subject. Non-limiting examples of antimicrobial agents include: anti-amikacin, gentamycin, kanamycin, neomycin, netilmicin, tobramycin, paromomycin, geldanamycin, herbimycin, loracarbef, ertapenem, doripenem, imipenem, meropenem, cefadroxil, cefazolin, cefalotin, cefalexin, cefaclor, cefamandole, cefoxitin, cefprozil, cefuroxime, cefixime, cefdinir, cefditoren, cefotaxime, cefpodoxime, ceftazidime, ceftibuten, ceftizoxime, ceftriaxone, cefepime, ceftobiprole, teicoplanin, vancomycin, telavancin, clindamycin, lincomycin, azithromycin, clarithromycin, dirithromycin, erythromycin, roxithromycin, troleandomycin, telithromycin, telithromycin, spectinomycin, aztronam, furazolidone, nitrofurantoin, nitrofurantoin, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, bacitracin, colistin, polymyxin B, ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfonamidochrysoidine, sulfacetamide, sulfadiazine, silver sulfadiazine, sulfamethizole, sulfamethoxazole, sulfanilamide, sulfasalazine, sulfisoxazole, trimethoprim, demeclocycline, doxycycline, minocycline, oxytetracycline, tetracycline, clofazimine, dapsone, capreomycin, cycloserine, ethambutol, ethionamide, isoniazid, pyrazinamide, rifampicin, rifabutin, rifapentine, streptomycin, arsphenamine, chloramphenicol, fosfomycin, fusidic acid, linezolid, metronidazole, mupirocin, platensimycin, quinupristin, rifaximin, thiamphenicol, and tinidazole.

By “admitted to a medical facility” is meant an order by a medical professional that the condition of a subject be monitored or that the subject be housed in a medical facility (e.g., for at least 12 hours, 1 day, 2 days, 3 days, 4 days, 5 days, 6 days, and one week). A person admitted to a medical facility may be admitted to a critical care or an intensive care unit.

By “amount” is meant either a mass or a molar quantity of a nucleic acid or peptide.

When a nucleic acid or peptide is herein referred to as being “absent” in an organism (e.g., human or bacteria), it is meant that the nucleic acid or peptide is not identically present in the genome of the organism as indicated by bioinformatics tools (e.g., BLAST or FASTA) for sequence comparison.

As used herein, “amplify” is meant the in vitro amplification of a nucleic acid of interest using, e.g., PCR and real-time PCR (e.g., quantitative PCR (qPCR)).

The term “effective,” used in the context of diagnostic assays, indicates that an oligonucleotide primer can be used, under a certain set of amplification conditions (e.g., pH, temperature, reaction time, number of amplification cycles, and buffer concentrations) to amplify a nucleic acid of interest.

By “endotoxin” is meant a compound (e.g., a lipid, protein, and/or carbohydrate) recognized by a mammal's (e.g., human, horse, cat, dog, monkey, baboon, mouse, and rat) immune system (e.g., cellular immune system, such as T-helper, cytotoxic T cells, NK cells, and/or PMN cells) that results in a local or systemic inflammatory response. For example, an endotoxin may be produced by a bacterium, a fungus, or a virus. One class of endotoxins is enterotoxins.

By the term “endotoxic” is meant an organism (e.g., a plant, bacterium, virus, fungi, or reptile) that produces one or more (e.g., two, three, four, or five) endotoxin(s).

By “infection-induced illnesses” is meant a disease state induced by an infection (e.g., a bacterial, fungal, or viral infection) in a subject. Non-limiting examples of infection-induced illnesses include organ failure (e.g., loss of function in one, two, three, or four organs), hypotension (e.g., a systolic blood pressure of less than 90 mm Hg and/or a diastolic blood pressure of less than 60 mm Hg), seizures, shock, increased heart rate (e.g., greater than 90 beats per minute), tachypnea (e.g., greater than 20 breaths per minute), and decreased arterial pressure of CO₂ (e.g., less than 4.3 kPa), and hemolytic-uremic syndrome. A subject having an infection-induced illness may also have an on-going bacterial infection or a subject having an infection-induced illness may not have an on-going bacterial infection (e.g., a subject that previously had an infection).

By “increased propensity to develop an infection-induced illness” is meant a subject that has at least a 10% (e.g., at least 15%, 20%, 25%, 30%, 35%, 40%, 45%, 50%, 55%, 60%, 65%, 70%, 75%, 80%, 85%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, 500%, 550%, 600%, 650%, 700%, 750%, 800%, 850%, 900%, 950%, 1000%, 1050%, 1100%, 1150%, 1200%, 1250%, 1300%, 1350%, 1400%, 1450%, 1500%, 1550%, 1600%, 1650%, 1700%, 1750%, 1800%, 1850%, 1900%, 1950%, 2000%, 2500%, 3000%, 3500%, 4000%, 4500%, or 5000%) increased risk of later developing (e.g., at least 3 hours, 6 hours, 9 hours, 12 hours, 15 hours, 18 hours, 21 hours, 24 hours, 27 hours, 30 hours, 36 hours, 39 hours, 42 hours, 45 hours, 48 hours, 3 days, 4 days, 5 days, 6 days, and 7 days) an infection-induced illness. For example, a subject may later develop an infection-induced illness following a microbial infection (e.g., bacterial infection)(e.g., an ongoing infection), potential exposure to an endotoxic bacterium, or a composition containing an endotoxin. An infection-induced illness as described herein may be an severe infection-induced illness.

By “initial presentation” is meant the first visit of a subject experiencing one or more symptoms to a medical professional. For example, the initial presentation may occur in a medical facility such as a hospital or health care clinic.

By “local infection” is meant an infection (e.g., bacterial, fungal, or viral) that is localized to a discrete tissue (one tissue) in a subject.

By “medical professional” is meant any person whose employment involves physical contact with a subject (e.g., a subject having a bacterial infection). Non-limiting examples of medical professionals include nurses, physician assistants, phlebotomists, lab technicians, and doctors, and the equivalent personnel in non-U.S. countries.

By “medical facility” is meant any location where a subject may come into physical contact with a medical professional. Non-limiting examples of a medical facility include a hospital or a health care clinic.

By “systemic infection” is meant an infection (e.g., bacterial, fungal, or viral) that is present in multiple (e.g., two or more) organs or tissues, or the entire body

By “measure” or “determine,” in the context of measuring or determining the amount of a nucleic acid in a sample, is meant quantitating a mass or a molar amount of the nucleic acid. Ways of measuring or determining nucleic acids are well known in the art and include, e.g., quantitative polymerase chain reaction (real-time qPCR). Non-limiting methods for measuring mitochondrial nucleic acid and microbial nucleic acid are described herein. The term measure may also be used in the context of measuring the amount of a peptide or polypeptide in a sample. Non-limiting methods for measuring mitochondrial peptides and microbial peptides are also described herein.

By “oligonucleotide primer” is meant an oligonucleotide, typically synthetic, that is useful for specifically binding and amplifying a sequence of interest by primer extension.

By “potential exposure” is meant the contact of a mammal (e.g., a human) to a substance or an organism (e.g., a bacterium, fungus, or virus) that may contain an endotoxin or may produce an endotoxin (e.g., produce an endotoxin during its growth cycle in a mammal), respectively. A mammal may be potentially exposed to such a substance or organism via inhalation, ingestion, and/or tactile contact.

By “ratio” is meant either a mass ratio or a molar ratio of nucleic acids or proteins. For a raw ratio obtained from measured nucleic acid, amounts may be normalized in various ways (e.g., for relative nucleic acid lengths, amplification biases, and other experimental considerations), before it is assessed against a cutoff value, used to determine a confidence level, or used to calculate the ratio. For a raw ratio obtained from measured proteins, amounts may be normalized to a control protein (e.g., the expression level of a house-keeping protein, such as β-actin), before it is assessed against a cutoff value, used to determine a confidence level, or used to calculate the ratio. Non-limiting examples of ratios of the amount of mitochondrial nucleic acid to the amount of microbial nucleic acid, in the context of the methods of the present invention, include a ratio of at least 25:1, 50:1, 75:1, 100:1, 150:1, 200:1, 250:1, 300:1, 350:1, 400:1, 450:1, 500:1, 550:1, 600:1, 650:1, 700:1, 750:1, 800:1, 850:1, 900:1, 950:1, 1000:1, 1050:1, 1100:1, 1150:1, 1200:1, 1250:1, 1300:1, 1350:1, 1400:1, 1450:1, 1500:1, 1600:1, 1700:1, 1800:1, 1900:1, or 2000:1. Non-limiting examples of ratios of the amount of mitochondrial peptides to the amount of microbial peptides include a ratio of at least 5.0:1, 6.0:1, 7.0:1, 8.0:1, 9.0:1, 10:1, 11:1, 12:1, 13:1, 14:1, 15:1, 16:1, 17:1, 18:1, 19:1, 20:1, 25:1, 26:1, 27:1, 28:1, 29:1, 30:1, 35:1, 40:1, 45:1, 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, 150:1, 155:1, 160:1, 165:1, 170:1, 175:1, 180:1, 185:1, 190:1, 195:1, or 200:1.

By “sample” is meant any specimen (e.g., blood, serum, plasma, urine, saliva, amniotic fluid, cerebrospinal fluid, tissue (e.g., placental or dermal), pancreatic fluid, chorionic villus sample, and cells) taken from a subject. Preferably, the sample is taken from a portion of the body affected by endotoxic shock.

By “severe” or “severity” is meant an increase by at least 5% (e.g., at least 10%, 15%, 20%, 25%, or 30%) in the intensity of one or more (e.g., two, three, or four) symptoms of a disease (e.g., an infection-induced illness) during the progression of a disease in a subject.

By “subject” is meant any animal (e.g., human, cat, dog, horse, monkey, mouse, rat, and rabbit).

By “symptoms of severe septic shock” is meant one or more (e.g., at least two, three, four, or five) physical manifestations that result in a mammal upon exposure to an endotoxin. Non-limiting examples of such symptoms include: hypotension (e.g., a systolic blood pressure of less than 90 mm Hg and/or a diastolic blood pressure of less than 60 mm Hg), vomiting, diarrhea, rash, seizures, shock, respiratory failure, altered body temperature (e.g., less than 36° C. or greater than 38° C.), increased heart rate (e.g., greater than 90 beats per minute), tachypnea (e.g., greater than 20 breaths per minute), decreased arterial pressure of CO₂ (e.g., less than 4.3 kPa), altered white blood count (e.g., less than 4,000 cells/mm³ or greater than 12,000 cells/mm³), increased histamine levels (e.g., greater than 60 ng/mL in blood), increased leukotriene B4 levels (e.g., greater than 30 pg/mL or greater than 35 pg/mL in blood), increased prostaglandin levels (e.g., greater than 3.0 ng/mL in blood), increased levels of pro-inflammatory cytokines (e.g., greater than 20 ng/mL TNF-α and/or greater than 10 pg/mL IL-6).

Other features and advantages of the invention will be apparent from the following description of the preferred embodiments thereof, and from the claims.

### BRIEF DESCRIPTION OF THE DRAWINGS

FIG. 1. The amounts of mtDNA and bacterial DNA in pancreatic juice were measured using real-time qPCR using primers for cytochrome B (mtDNA) and 16S rRNA (bDNA). Water was used as a control.

FIG. 2. The amounts of mtDNA and bacterial DNA in pancreatic juice were measured using real-time qPCR using primers for cytochrome B (mtDNA) and 16S rRNA (bDNA). Water was used as a control.

FIG. 3. The amounts of mtDNA and bacterial DNA in pancreatic fluid in control rats and a rat model of pancreatitis were measured using real-time qPCR. The fold-increase in mtDNA and bacterial DNA in pancreatic fluid relative to control is depicted.

FIG. 4. The amounts of mtDNA and bacterial DNA were measured in the serum of baboons using qPCR 0 to 96 hours after intravenous administration of shiga toxin-1. Primers for mitochondrial cytochrome B (mtDNA) and bacterial 16S rRNA (bDNA) were used.

FIG. 5. The amounts of mtDNA and bacterial DNA were measured in the serum of baboons using qPCR 0 to 48 hours after 1-hour infusion of a sublethal dose of E. coli. Primers for mitochondrial cytochrome B (mtDNA) and bacterial 16S rRNA (bDNA) were used. Both bacterial and mitochondrial DNAs appear in the bloodstream after E. coli infusion. This demonstrates a septic insult to the host. After cessation of the infusion however, both the 16s (infection) signal and the CytB (injury) signal dissipate and the animals recover.

FIG. 6. The amounts of mtDNA and bacterial DNA were measured in the serum of baboons using qPCR 0 to 96 hours after 1-hour infusion of a dose of B. anthracis containing a mutation in the gene for anthrax toxin. Baboons either received no further treatment (B. anthracis) or received a dose of activated protein C at the time of B. anthracis infusion (B. anthracis plus APC). Primers for mitochondrial cytochrome B (mtDNA) and bacterial 16S rRNA (bDNA) were used.

FIG. 7. The amounts of mtDNA and bacterial DNA were measured in the serum of baboons using qPCR 0 to 96 hours after 1-hour infusion of a dose of B. anthracis containing a mutation in the gene for anthrax toxin. Baboons either received no further treatment (B. anthracis) or received a dose of activated protein C at the time of B. anthracis infusion (B. anthracis plus APC). Primers for mitochondrial cytochrome B (mtDNA) and bacterial 16S rRNA (bDNA) were used.

FIG. 8 is a graph showing the results of blood cultures of 4 baboons per group before, during, and after anthrax infusion. All animals received levofloxacin (7 mg/kg) four hours after the start of bacterial infusion and daily thereafter. Grey bars are without aPC pre-treatment, black bars are with aPC pre-treatment, and there was no significant difference between groups. Colony counts varied according to the loading dose. For a 1E8 CFU/kg challenge, colony counts were near 1E4 CFU/mL at T=2 hours and 100 CFU/mL at T=4 hours. Colony counts on blood sampled between days 2 to 7 were consistently negative.

FIG. 9A is a graph showing plasma bDNA peaks over the time course indicated after Anthrax administration. FIG. 9B is a graph showing plasma mtDNA peaks over the time course indicated after Anthrax administration. Rescue with aPC prompts reduction in mtDNA and survival. Without rescue from SIRS, mtDNA levels remain high even after bacteremia is gone. This shows continued SIRS and all those animals die. bDNA cleared after 48 hr, with a peak near 10 hr. Bacteremia induced over 2 hr and treated with antibiotics at 2 hr. aPC rescue does not affect plasma bDNA level.

FIG. 10 is a graph showing Baboons given Shiga toxin 1 get organ failure and die. The injury is completely sterile. The animals show systemic injury in the form of mtDNA but no evidence in the form of bDNA in their plasma.

FIGS. 11A and 11B are graphs showing that aPC alters the relationship between inflammatory stimuli and respiratory rat (respiratory rate versus markers for PAMPs and DAMPs). Respiratory rate does not vary with bDNA (FIG. 11A), rather, without aPC, respiratory rate simply increases over time. In distinction, respiratory rate does vary with mtDNA concentration, without aPC (FIG. 11B). In both cases aPC prevents tachypnea.

FIGS. 12A-12D are graphs showing the results of kidney and liver function assays. Plasma transaminases (ALT, AST) were studied as markers for hepatocellular injury in sepsis and SIRS over the first 24 hours (FIGS. 12A and 12B, respectively). Bilirubin showed a similar pattern. Blood urea nitrogen (BUN) and creatinine were studied similarly as markers for kidney injury (FIGS. 12C and 12D, respectively). All these markers of septic organ injury showed dramatic increases after anthrax infusion with the increases beginning around 6 hours and continuing through 24 hours. Thus, liver and kidney function were progressively compromised in sepsis due to B. anthracis.

FIGS. 13A-13C are graphs showing the results of hematologic function assays. FIG. 12A shows that anthrax sepsis led to a rapid and precipitous fall in fibrinogen during the period of bacteremia itself, followed by slow restitution. FIGS. 13B and 13C show that hematocrit and hemoglobin, respectively, appear to rise early in sepsis reflecting capillary leak syndrome and hemo-concentration.

FIGS. 14A and 14B are graphs showing changes in fibrinogen levels versus markers for PAMPS and DAMPS. In FIG. 14A, fibrinogen presents as a function of sepsis, represented by bDNA. Regardless of treatment, mtDNA concentration does not correlate to plasma fibrinogen level (FIG. 14B). However, DIC (as indicated by defibrination), appears strongly linked to bacteremia (as indicated by bDNA). Thus, the presence of bacteria appears specifically to induce DIC, whereas the inflammatory response to tissue injury does not cause DIC, even when it is initiated by bacteremia.

FIG. 15 is a graph showing the plasma levels of mitochondrial Cyto-B DNA (blue) and bacterial 16S-DNA (red) over time in a subject.

### DETAILED DESCRIPTION

Subjects with infections (e.g., local or systemic bacterial infections) or subjects previously having an infection (e.g., bacterial infection) may develop one or more infection-induced illness(es). There is a need for methods of identifying subjects that have an increased risk of later developing an infection-induced illness. The invention provides both methods and kits for identifying whether a subject has an increased likelihood or propensity to later develop an infection-induced illness, as well as methods and kits for identifying patients that have an increased risk of later developing a severe infection-induced illness. The invention further provides methods of treating a subject with a microbial (e.g., a bacterial) infection.

### Diagnostic Methods

The likelihood of a subject (e.g., a human, cat, dog, horse, rabbit, mouse, monkey, or rat) to develop one or more (e.g., two, three, or four) infection-induced illness(es) or to develop one or more severe infection-induced illness(es) in the future may be determined by: (a) measuring the amount of microbial (e.g., bacterial) nucleic acid or peptide in a sample from the subject; (b) measuring the amount of mitochondrial nucleic acid or peptide in the sample; and (c) determining whether the subject has an increased likelihood (e.g., at least a 5%, 10%, 15%, 20%, 25%, 30%, 35%, 40%, 45%, 50%, 55%, 60%, 65%, 70%, 75%, 80%, 85%, 90%, 95%, 100%, 110%, 120% 130%, 140%, 150%, 160%, 170%, 180%, 190%, 200%, 210%, 220%, 230%, 240% 250%, 260%, 270%, 280%, 290%, 300%, 350%, 400%, 450%, 500%, 550%, 600%, 650%, 700%, 750%, 800%, 850%, 900%, 950%, 1000%, 1100%, 1200%, 1300%, 1400%, 1500%, 1600%, 1700%, 1800%, 1900%, 2000%, 2100%, 2200%, 2300%, 2400%, 2500%, 2600%, 2700%, 2800%, 2900%, or 3000% increased likelihood) of later developing (e.g., at least 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 24 hours, 28 hours, 32 hours, 36 hours, 40 hours, 44 hours, 48 hours, 3 days, 4 days, 5 days, 6 days, or 7 days after initial presentation to a medical professional) an infection-induced illness (e.g., organ failure) by comparing the amount of microbial (e.g., bacterial) nucleic acid or peptide measured in the sample with the amount of mitochondrial nucleic acid or peptide measured in the sample, where an increased ratio of the amount of mitochondrial nucleic acid or peptide to the amount of microbial (e.g., bacterial) nucleic acid or peptide indicates a subject with an increased likelihood of later developing one or more infection-induced illness(es) or indicates a subject as having an increased propensity to develop one or more severe infection-induced illness(es) in the future.

Subjects may also be identified as having an increased propensity (e.g., at least a 5%, 10%, 15%, 20%, 25%, 30%, 35%, 40%, 45%, 50%, 55%, 60%, 65%, 70%, 75%, 80%, 85%, 90%, 95%, 100%, 110%, 120% 130%, 140%, 150%, 160%, 170%, 180%, 190%, 200%, 210%, 220%, 230%, 240% 250%, 260%, 270%, 280%, 290%, 300%, 350%, 400%, 450%, 500%, 550%, 600%, 650%, 700%, 750%, 800%, 850%, 900%, 950%, 1000%, 1100%, 1200%, 1300%, 1400%, 1500%, 1600%, 1700%, 1800%, 1900%, 2000%, 2100%, 2200%, 2300%, 2400%, 2500%, 2600%, 2700%, 2800%, 2900%, or 3000% increased propensity) to later develop at least 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 24 hours, 28 hours, 32 hours, 36 hours, 40 hours, 44 hours, 48 hours, 3 days, 4 days, 5 days, 6 days, or 7 days after initial presentation to a medical professional) one or more infection-induced illness(es) or one or more severe infection-induced illness(es) (e.g., organ failure) by: measuring the amount of microbial (e.g., bacterial) nucleic acid or peptide in a sample from the subject; measuring the amount of a mitochondrial nucleic acid or peptide in the sample; and comparing the amount of microbial (e.g., bacterial) nucleic acid or peptide measured with the amount of mitochondrial nucleic acid or peptide measured, where an increased ratio of the amount of mitochondrial nucleic acid or peptide to the amount of microbial (e.g., bacterial) nucleic acid or peptide identifies a subject as having an increased propensity to later develop one or more infection-induced illness(es) or an increased propensity to later develop one or more severe infection-induced illness(es).

The invention also provides methods of diagnosing one or more infection-induced illness(es) in a subject or predicting the future severity of one or more infection-induced illness(es) requiring the steps of: measuring the amount of microbial (e.g., bacterial) nucleic acid or peptide in a sample from the subject; measuring the amount of a mitochondrial nucleic acid or peptide in the sample; and comparing the amount of microbial (e.g., bacterial) nucleic acid or peptide measured with the amount of mitochondrial nucleic acid or peptide measured, where an increased ratio of the amount of mitochondrial nucleic acid or peptide to the amount of microbial (e.g., bacterial) nucleic acid or peptide indicates that the subject has one or more infection-induced disorders or indicates that the one or more infection-induced illness(es) may be severe in the future. Subjects identified or diagnosed as having one or more infection-induced illness(es) (e.g., using the methods described herein) may be admitted to a medical facility or treated with one or more antimicrobial or one or more anti-inflammatory agents (e.g., e.g., anti-FPR1 antibodies, cyclosporine H, activated protein C, chloroquine, and/or anti-TLR9 antibodies).

Infection-induced illness include, but are not limited to, organ failure, hypotension, seizures, shock, increased heart rate, tachypnea, decreased arterial pressure of CO₂, and hemolytic-uremic syndrome. Infection-induced illnesses may result in death. The subject in these methods may have an infection (e.g., a bacterial infection, such as a local or systemic infection). The subject may also not demonstrate or present with any symptoms of severe septic shock as described herein. The subject may have a bacterial infection caused by one or more bacteria selected from the group of: Bacillus athracis, Vibrio cholera, Bordetella pertussis, Eschericia coli, Clostridium tetani, Clostridium perfringes, Clostridium difficile, Clostridium botulinum, Listeria monocytogenes, Streptococcus spp., Staphylococcus aureus, Mycobacterium tuberculosis, Corynebacterium diphtheria, Shigella dysenteriae, Pseudomonas aeriginosa, and Bacillus thuringiensis.

The sample may be obtained from the subject within 24 hours (e.g., within 20 hours, 16 hours, 12 hours, 8 hours, 4 hours, 3 hours, 2 hours, or 1 hour) of an initial presentation of the subject to a medical professional. The sample may also be obtained from a subject at least 24 hours (e.g., at least 48 hours, 3 days, 4 days, 5 days, 6 days, or one week) after an initial presentation of the subject to a medical professional. The sample may also be obtained from a subject that has already been admitted to a medical facility.

The sample may also be obtained from subject that has been potentially exposed to an endotoxic bacterium or a composition containing an endotoxin (e.g., anthrax toxin or shiga toxin). In such instances, the sample may be obtained from the subject within 3 to 24 hours (e.g., 3 to 20 hours, 3 to 16 hours, 3 to 12 hours, or 3 to 8 hours) after a potential exposure to an endotoxic bacterium or a composition containing an endotoxin.

The sample may represent any specimen obtained from a subject. Non-limiting examples of a sample include blood, serum, plasma, urine, saliva, amniotic fluid, cerebrospinal fluid, tissue (e.g., placental or dermal), pancreatic fluid, chorionic villus sample, and cells taken from a subject. The sample may be taken from a portion of the body affected by infection (e.g., local or systemic bacterial infection). The sample may be obtained by intravenous puncture, intraarterial puncture, lumbar puncture, amniopuncture, urine sample collection, sputum collection, or biopsy.

In the provided methods, the subject may also not have (present with) symptoms of a severe septic shock. Non-limiting examples of such symptoms include: altered body temperature (e.g., less than 36° C. or greater than 38° C.), increased heart rate (e.g., greater than 90 beats per minute), tachypnea (e.g., greater than 20 breaths per minute), decreased arterial pressure of CO₂ (e.g., less than 4.3 kPa), altered white blood count (e.g., less than 4,000 cells/mm³ or greater than 12,000 cells/mm³), increased histamine levels (e.g., greater than 60 ng/mL in blood), increased leukotriene B4 levels (e.g., greater than 30 pg/mL or greater than 35 pg/mL in blood), increased prostaglandin levels (e.g., greater than 3.0 ng/mL in blood), increased levels of pro-inflammatory cytokines (e.g., greater than 20 ng/mL TNF-α and/or greater than 10 pg/mL IL-6).

The amount of mitochondrial nucleic acid or peptide present in a sample may be measured using standard techniques known in the art. For example, mitochondrial nucleic acids may be measured using quantitative techniques such as real-time qPCR using primers designed to specifically amplify nucleic acid sequences present in the mitochondrial genome. In desirable embodiments, the mitochondrial nucleic acid sequence amplified by qPCR is unique to the mitochondrial genome and is not present in the nuclear genome of the subject (e.g., a mammal). For example, mitochondrial nucleic acid sequences that may be measured in the above methods include cytochrome B, cytochrome C oxidase subunit III, and NADH dehydrogenase. For humans, amplification of mitochondrial cytochrome B may be performed using the forward primer 5′-atgaccccaatacgcaaaat-3′ (SEQ ID NO: 1) and the reverse primer 5′-cgaagtttcatcatgcggag-3′ (SEQ ID NO: 2), amplification of mitochondrial cytochrome C oxidase subunit III may be performed using the forward primer forward primer 5′-atgacccaccaatcacatgc-3′ (SEQ ID NO: 15) and the reverse primer 5′-atcacatggctaggccggag-3′ (SEQ ID NO: 16), and amplification of mitochondrial NADH dehydrogenase may be performed using the forward primer 5′-atacccatggccaacctcct-3′ (SEQ ID NO: 5) and the reverse primer 5′-gggcctttgcgtagttgtat-3′ (SEQ ID NO: 6). Additional primers may be used to amplify additional mitochondrial nucleic acid sequences, including but not limited to nucleic acid sequences containing a sequence at least 95% (e.g., at least 96%, 97%, 98%, 99%, or even 100% identical) to NADH dehydrogenase subunit I (nucleotides 3308-4264 of SEQ ID NO: 21), NADH dehydrogenase subunit II (nucleotides 4471-5512 of SEQ ID NO: 21), NADH dehydrogenase subunit III (nucleotides 10,060 to 10,405 of SEQ ID NO: 21), NADH dehydrogenase subunit IV (nucleotides 10,761 to 12,138 of SEQ ID NO: 21), NADH-ubiquinone oxidoreductase chain 4L (nucleotides 10,471 to 10,767 of SEQ ID NO: 21), NADH dehydrogenase subunit V (nucleotides 12,338 to 14,149 of SEQ ID NO: 21), NADH dehydrogenase VI (nucleotides 14,150 to 14,674 of SEQ ID NO: 21), cytochrome B (nucleotides 14,748 to 15,882 of SEQ ID NO: 21), cytochrome C oxidase subunit I (nucleotides 5905 to 7446 of SEQ ID NO: 21), cytochrome C oxidase subunit II (nucleotides 7587 to 8270 of SEQ ID NO: 21), cytochrome C oxidase subunit III (nucleotides 9208 to 9988 of SEQ ID NO: 21), ATP synthase FO subunit VI (nucleotides 8528 to 9208 of SEQ ID NO: 21), and ATP synthase subunit VIII (nucleotides 8,367 to 8,573 of SEQ ID NO: 21). The levels of the measured mitochondrial nucleic acid samples may be normalized to a standard or a reference in the real-time PCR experiment. For example, a real-time PCR standard curve may be created to quantify the mtDNA concentration by using purified mtDNA. Methods for the isolation of mtDNA are described in the Examples. The threshold level (Ct) for amplification in real-time PCR may be set at 20, 25, 30, 35, or 40 cycles for statistical purposes. Desirably, the threshold level for amplification in real-time PCR is set at 30 or 40 cycles. Mitochondrial RNA may also be measured as the mitochondrial nucleic acid in the above methods. In such experiments, a first step of synthesis of a cDNA copy of the mitochondrial RNA is performed using reverse transcriptase prior to amplification in a real-time PCR experiment.

Mitochondrial peptides (N-formylated peptides) may be measured using standard methods known in the art. For example, expression of mitochondrial proteins (e.g., NADH dehydrogenase subunit I, NADH dehydrogenase subunit II, NADH dehydrogenase subunit III, NADH dehydrogenase subunit IV, NADH-ubiquinone oxidoreductase chain 4L, NADH dehydrogenase subunit V, NADH dehydrogenase subunit VI, cytochrome B, cytochrome C oxidase subunit I, cytochrome C oxidase subunit II, cytochrome C oxidase subunit III, ATP synthase FO subunit VI, and ATP synthase subunit VIII) may be measured using ELISA assays, Western blotting assays, or protein array assays. The relative level of expression of mitochondrial proteins may be compared to the levels of purified mitochondrial proteins or to other control proteins present in the sample. A number of antibodies that specifically bind mitochondrial peptides are commercially available.

Similarly, the amount of microbial (e.g., bacterial, fungal, or viral) nucleic acid or peptides in a sample may be measured using standard techniques known in the art. For example, bacterial nucleic acids may be measured quantitative techniques such as real-time PCR using primers designed to specifically amplify sequences present in the bacterial genome. In desirable embodiments, the bacterial nucleic acid that is amplified is common to all species of bacteria, but not expressed in a mammalian cell. For example, specific sequences in 16S rRNA are conserved among many bacterial species and may be used to design primers that amplify 16S rRNA from several different species of bacteria using real-time PCR. In other examples, the primers used to quantitate the bacterial nucleic acid are designed to amplify 16S rRNA from a single species of bacteria using real-time PCR (see, for e.g., the primers described in WO 08/03957, herein incorporated by reference). In desirable embodiments, the bacterial nucleic acid sequence amplified by real-time PCR is unique to bacteria and is not expressed in a mammalian cell. One set of primers that may be used to amplify 16S rRNA from a variety of bacterial species are 5′-tgtagcggtgaaatgcgtaga-3′ (SEQ ID NO: 13) and 5′-ccagggtatctaatcctgtttg-3′ (SEQ ID NO: 14). The levels of the measured bacterial nucleic acid may be normalized to a standard or reference in the real-time PCR experiment. For example, a real-time PCR standard curve may be created to quantify the bacterial nucleic acid concentration by using purified 16S rRNA. Methods for the isolation of 16S rRNA for use as a standard control are known in the art. The threshold level (Ct) for amplification in real-time PCR may be set at 20, 25, 30, 35, or 40 cycles for statistical purposes. Desirably, the threshold level (Ct) for amplification in real-time PCR is set at least 20 or at least 30 cycles. As noted above, prior to direct use in real-time PCR, 16S rRNA or bacterial RNA must first be reverse transcribed into a cDNA prior to its amplification in real-time PCR.

The sample obtained from the subject may need to be treated in order to release the bacterial DNA from any bacteria present in the sample. Methods for the use of a microfluidic device for lysis of bacterial cells in a sample are described in WO 09/002580 and U.S. 2007/0015179, incorporated by reference in its entirety. Additional methods for bacterial lysis in a biological sample are known in the art and include without limitation: alkaline lysis (provided in a number of commercially available kits), lysozyme treatment, physical disruption (e.g., French press), or combination thereof. Such lysis methods may be used prior to the subsequent amplification of the nucleic acids using PCR-based techniques (e.g., real-time PCR).

Bacterial peptides may also be measured using standard methods known in the art. For example, expression of bacterial proteins may be measured using ELISA assays, Western blotting assays, or protein array assays. The relative level of expression of bacterial proteins may be compared to the levels of purified bacterial proteins or to other control proteins present in the sample. A number of antibodies that specifically bind bacterial peptides are commercially available.

Primers for the amplification of a fungal or viral nucleic acid (e.g., DNA or RNA) may also be designed using sequences known in the art. Similarly, assays to measure the level of a fungal or viral peptide may be performed using commercially available antibodies or antibodies generated using monoclonal antibody technology.

A ratio (increased ratio) of the amount of mitochondrial nucleic acid or peptide to the amount of microbial (e.g., bacterial) nucleic acid or peptide present in a sample that indicates that a subject having an increased likelihood or propensity of later developing one or more infection-induced illness(es) or that a subject has one or more infection-induced illness(es) may be a ratio of at least 5.0:1, 6.0:1, 7.0:1, 8.0:1, 9.0:1, 10:1, 11:1, 12:1, 13:1, 14:1, 15:1, 16:1, 17:1, 18:1, 19:1, 20:1, 25:1, 26:1, 27:1, 28:1, 29:1, 30:1, 35:1, 40:1, 45:1, 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, 150:1, 155:1, 160:1, 165:1, 170:1, 175:1, 180:1, 185:1, 190:1, 195:1, 200:1, 250:1, 300:1, 350:1, 400:1, 450:1, 500:1, 550:1, 600:1, 650:1, 700:1, 750:1, 800:1, 850:1, 900:1, 950:1, 1000:1, 1050:1, 1100:1, 1150:1, 1200:1, 1250:1, 1300:1, 1350:1, 1400:1, 1450:1, 1500:1, 1600:1, 1700:1, 1800:1, 1900:1, or 2000:1. The term increased ratio may be compared related to a threshold ratio (e.g., one of the ratios listed above) or the measured ratio in a control subject (e.g., a subject without infection or without an infection-induced illness). The determined ratio may represent a mass ratio or a molar ratio of the nucleic acids or proteins. For a raw ratio obtained from a measured nucleic acid, the amounts may be normalized in various ways (e.g., for relative nucleic acid lengths, amplification biases, and other experimental considerations), before it is assessed against a cutoff value or used to determine a confidence level or to calculate the ratio. Similarly, for a raw ratio obtained from a measured protein, the amounts may be normalized to another protein present in the sample (e.g., normalized to the level of a house-keeping gene such as actin), before it is assessed against a cutoff value or used to determine a confidence level or to calculate the ratio.

### Methods of Treatment

Methods of treating a subject with a microbial (e.g., bacterial) infection are provided that require: measuring the amount of microbial (e.g., bacterial) nucleic acid or peptide in a sample from the subject; measuring the amount of a mitochondrial nucleic acid or peptide in the sample; comparing the amount of microbial (e.g., bacterial) nucleic acid or peptide measured to the amount of mitochondrial nucleic acid or peptide; and administering to the subject having an increased ratio of the amount of mitochondrial nucleic acid or peptide one or more anti-inflammatory agent(s) (e.g., e.g., anti-FPR1 antibodies, cyclosporine H, activated protein C, chloroquine, and/or anti-TLR9 antibodies) and one or more antimicrobial agent(s). In one example, the subject with an increased ratio of mitochondria nucleic acids or peptide to microbial (e.g., bacterial) nucleic acids or peptides is treated with one or more doses of one or more antimicrobial agents prior to the administration of one or more doses of one or more anti-inflammatory agents. For example, the subject is administered one or more anti-inflammatory agents (e.g., e.g., anti-FPR1 antibodies, cyclosporine H, activated protein C, chloroquine, and/or anti-TLR9 antibodies) at least 12 hours (e.g., at least 16 hours, 24 hours, 32 hours, 40 hours, 48 hours, 3 days, 4 days, 5 days, 6 days, or 1 week) after administration of the one or more antimicrobial agents.

The methods may result in a reduction in the likelihood of developing one or more infection-induced illness(es) as described herein. The subject may already be admitted to a medical facility, not yet admitted to a medical facility, or may have previously been diagnosed as having a bacterial infection (e.g., local or systemic infection). The subject may also have been potentially exposed to an endotoxic bacterium or a composition containing an endotoxin (e.g., shiga toxin or anthrax toxin). For example, a sample may be obtained from a subject within 3 to 24 hours after a potential exposure to an endotoxic bacterium or a composition containing an endotoxin.

A variety of different samples may be obtained from the subjects using any of the above described methods. The sample may be obtained from the subject at a variety of different time points as described above.

A ratio (increased ratio) of the amount of mitochondrial nucleic acid or peptide to the amount of microbial (e.g., bacterial) nucleic acid or peptide present in a sample that indicates that a subject should administered one or more antimicrobial agents and one or more anti-inflammatory agents may be a ratio of at least 5.0:1, 6.0:1, 7.0:1, 8.0:1, 9.0:1, 10:1, 11:1, 12:1, 13:1, 14:1, 15:1, 16:1, 17:1, 18:1, 19:1, 20:1, 25:1, 26:1, 27:1, 28:1, 29:1, 30:1, 35:1, 40:1, 45:1, 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, 150:1, 155:1, 160:1, 165:1, 170:1, 175:1, 180:1, 185:1, 190:1, 195:1, 200:1, 250:1, 300:1, 350:1, 400:1, 450:1, 500:1, 550:1, 600:1, 650:1, 700:1, 750:1, 800:1, 850:1, 900:1, 950:1, 1000:1, 1050:1, 1100:1, 1150:1, 1200:1, 1250:1, 1300:1, 1350:1, 1400:1, 1450:1, 1500:1, 1600:1, 1700:1, 1800:1, 1900:1, or 2000:1. The term increased ratio may be compared related to a threshold ratio (e.g., one of the ratios listed above) or the measured ratio in a control subject (e.g., a subject bacterial infection or an infection-induced illness). The determined ratio may represent a mass ratio or a molar ratio of the nucleic acids or proteins. For a raw ratio obtained from a measured nucleic acid, the amounts may be normalized in various ways (e.g., for relative nucleic acid lengths, amplification biases, and other experimental considerations), before it is assessed against a cutoff value or used to determine a confidence level or to calculate the ratio. Similarly, for a raw ratio obtained from a measured protein, the amounts may be normalized to another protein present in the sample (e.g., normalized to the level of a house-keeping gene such as (β-actin), before it is assessed against a cutoff value or used to determine a confidence level or to calculate the ratio. All of the methods for measuring the amount of a microbial (e.g., bacterial) nucleic acid or peptide and the amount of a mitochondrial nucleic acid or peptide described above may be used in the treatment methods without limitation. For example, an increased ratio indicating an increased risk of later developing one or more infection-induced illness(es) or an increased propensity to later develop a severe infection-induced illness(es) may be >1000:1, >800:1, or >700:1.

The invention further provides methods of administering to a subject one or more (e.g., two, three, four, or five) anti-inflammatory agents (e.g., e.g., anti-FPR1 antibodies, cyclosporine H, activated protein C, chloroquine, and/or anti-TLR9 antibodies) to a subject indicated as having an increased propensity to later develop one or more infection-induced illness(es).

Non-limiting examples of antimicrobial agents (e.g., anti-bacterial, anti-fungal, and/or anti-protozoan agents) that may not be administered or administered at a decreased dosage include: amikacin, gentamycin, kanamycin, neomycin, netilmicin, tobramycin, paromomycin, geldanamycin, herbimycin, loracarbef, ertapenem, doripenem, imipenem, meropenem, cefadroxil, cefazolin, cefalotin, cefalexin, cefaclor, cefamandole, cefoxitin, cefprozil, cefuroxime, cefixime, cefdinir, cefditoren, cefotaxime, cefpodoxime, ceftazidime, ceftibuten, ceftizoxime, ceftriaxone, cefepime, ceftobiprole, teicoplanin, vancomycin, telavancin, clindamycin, lincomycin, azithromycin, clarithromycin, dirithromycin, erythromycin, roxithromycin, troleandomycin, telithromycin, telithromycin, spectinomycin, aztronam, furazolidone, nitrofurantoin, nitrofurantoin, amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, ticarcillin, bacitracin, colistin, polymyxin B, ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin, mafenide, sulfonamidochrysoidine, sulfacetamide, sulfadiazine, silver sulfadiazine, sulfamethizole, sulfamethoxazole, sulfanilamide, sulfasalazine, sulfisoxazole, trimethoprim, demeclocycline, doxycycline, minocycline, oxytetracycline, tetracycline, clofazimine, dapsone, capreomycin, cycloserine, ethambutol, ethionamide, isoniazid, pyrazinamide, rifampicin, rifabutin, rifapentine, streptomycin, arsphenamine, choramphenicol, fosfomycin, fusidic acid, linezolid, metronidazole, mupirocin, platensimycin, quinupristin, rifaximin, thiamphenicol, and tinidazole.

Anti-inflammatory agents that may be administered in the methods of treatment include without limitation: inflammatory agents (e.g., ibuprofen, naproxen, fenoprofen, ketoprofen, flurbiprofen, oxaprozin, indomethacin, sulindac, etodolac, ketorolac, diclofenac, nabumetone, piroxicam, meloxicam, tenoxicam, droxicam, lornoxicam, isoxicam, mefenaic acid, meclofenamic acid, tolfenamic acid, celecoxib, rofecoxib, valdecoxib, parecoxib, lumiracoxib, etoricoxib, firoxocib, nimesulide, and licofelone), immunosuppressive agents (e.g., methotrexate, azathioprine, basiliximab, daclizumab, cyclosporine, tacrolimus, sirolimus, voclosporin, infliximab, etanercept, adalimumab, mycophenolic acid, fingolimod, pimecrolimus, thalidomide, lenalidomide, anakinra, deferolimus, everolimus, temsirolimus, zotarolimus, biolimus A9, and elsilimomab), corticosteroids (e.g., hydrocortisone, hydrocortisone acetate, cortisone acetate, tixocortol pivalate, prednisolone, methylprednisolone, prednisone, triamcinolone acetonide, triamcinolone alcohol, mometasone, amcinonide, budesonide, desonide, fluocinonide, fluocinolone acetonide, halcinonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate, and fluprednidene acetate), cyclosporine H, anti-FPR1 antibodies, and activated protein C. Desirably, the subject identified as having a increased propensity to develop an infection-induced illness is administered cyclosporine H, anti-FPR1, CpG oligodeoxynucleotides (e.g., CpG deoxynucleotides that contain one or more modified nucleotides, such as LNA) and/or activated protein C.

One or more anti-inflammatory agent(s) may be administered to the subject at a dose of 0.1 mg to 10 mg, 1 mg to 50 mg, 1 mg to 100 mg, 50 mg to 100 mg, 50 mg to 200 mg, 100 mg to 200 mg, 100 mg to 500 mg, 250 mg to 500 mg, 400 mg to 800 mg, 500 mg to 1 g, 600 mg to 1.5 g, 800 mg to 1.2 g, 1.0 g to 1.5 g, 1.5 g to 2.0 g. The amount and frequency of administration will dependent on several factors that may be determined by a physician including the mass, sex, disease state, and age of the subject. For example, a subject may be administered one or more anti-inflammatory agents continuously, every 2 hours, every 3 hours, every 4 hours, every 5 hours, every 6 hours, every 8 hours, every 10 hours, every 12 hours, once a day, two times a day, three times a day, four times a day, once a week, twice a week, three times a week, four times a week, five times a week, six times a week, seven times a week, biweekly, monthly, or bimonthly. The one or more anti-inflammatory agent(s) may be administered by any known means of administration, e.g., orally, intravenously, subcutaneously, and intaarterially. The subject may be monitored by a physician during the treatment for the development of symptoms of infection-induced illness or infection (e.g., bacterial infection). In response to the development of such symptoms, the physician may administer an increased dosage of one or more anti-inflammatory agents or increase the frequency of administration of such anti-inflammatory agents.

### Kits

The invention also provides kits containing one or more (e.g., two, four, six, or eight) oligonucleotide primers effective (e.g., capable of hybridizing to a microbial (e.g., bacterial) nucleic acid) for the amplification of a microbial (e.g., bacterial) nucleic acid, one or more (e.g., two, four, six, or eight) oligonucleotide primers effective (e.g., capable of hybridizing to a mitochondrial nucleic acid) for the amplification of mitochondrial nucleic acids, and instructions for using these primers to determine the likelihood that a subject will develop one or more infection-induced illness(es), to identify a subject that has an increased propensity to later develop one or more infection-induced illness(es) or one or more severe infection-induced illness(es), to determine whether a subject has an infection-induced illness, to predict the future severity of one or more infection-induced illness(es), to diagnose a subject as having one or more infection-induced illness(es), or to identify a subject at increased risk for developing a one or more infection-induced illness(es) in the future. These kits may include, without limitation, any of the nucleic acid primers described above for use in the diagnostic methods. The kits may further include control nucleic acid sequences for use in real-time PCR including purified mtDNA and/or bacterial 16S rRNA. The instructions provided with the kits may describe how to calculate the specific ratio of the amount of mitochondrial nucleic acid to the amount of microbial (e.g., bacterial) nucleic acid (e.g., exemplary methods for the calculation of the ratio are described herein). The instructions may also describe the comparison of the calculated ratio to a specific threshold value or a ratio measured from a control sample (e.g., a subject that does not have an infection (e.g., bacterial infection) or an infection-induced illness).

The invention also provides kits containing one or more (e.g., two, three, or four) antibodies that specifically bind to one or more microbial (e.g., bacterial) peptides, one or more (e.g., two, three, or four) antibodies that specifically bind one or more mitochondrial peptides, and instructions for using these antibodies to determine the likelihood that a subject will develop one or more infection-induced illness(es), to identify a subject that has an increased propensity to later develop one or more infection-induced illness(es) or one or more severe infection-induced illness(es), to determine whether a subject has an infection-induced illness, to predict the future severity of one or more infection-induced illness(es), to diagnose a subject as having one or more infection-induced illness(es), or to identify a subject at increased risk for developing a one or more infection-induced illness(es) in the future. The kits may further include control mitochondrial peptides and microbial peptides for use in assays (e.g., for use in generating a standard curve for ELISA assays). The instructions provided with the kits may describe how to calculate the specific ratio of the amount of mitochondrial peptide(s) to the amount of microbial (e.g., bacterial) peptide(s) (e.g., exemplary methods for the calculation of the ratio are described herein). The instructions may also describe the comparison of the calculated ratio to a specific threshold value or a ratio measured from a control sample (e.g., a subject that does not have an infection (e.g., bacterial infection) or an infection-induced illness).

### EXAMPLES

The features and other details of the invention will now be more particularly described and pointed out in the following examples describing preferred techniques and experimental results. These examples are provided for the purpose of illustrating the invention and should not be construed as limiting.

### Example 1

### Methods for qPCR Amplification of Bacterial DNA and Mitochondrial DNA

Exemplary methods for qPCR of bacterial DNA and mitochondrial DNA from a sample are described below. DNA may be prepared from 200 μL plasma using QIAamp DNA Blood Mini kit from Qiagen (Valencia, Calif.) according to the manufacturer's protocol. The same amount of DNA may be used for each Real Time PCR reaction using SYBR Green Master Mix (Applied Biosystems, Foster City, Calif.) by Mastercycler ep realplex from Eppendorf (Foster City, Calif.). Primers for mtDNA markers Cyt B, COX III, NADH, and the nuclear DNA marker, GAPDH, may be synthesized by a commercial manufacturer (e.g., Invitrogen) (Table 4). A standard curve may be created to quantify mtDNA concentration using purified mtDNA with Cyt B as the target. All data analysis may be performed according to the manufacturer's protocol.

### Example 2

### Pancreatitis is Associated with an Increased Level of mtDNA in Pancreatic Fluid

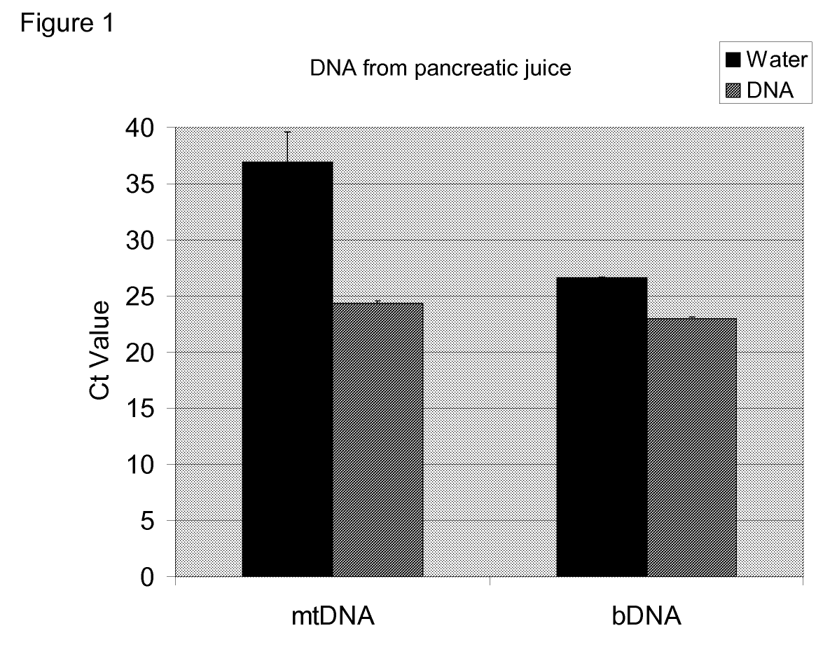

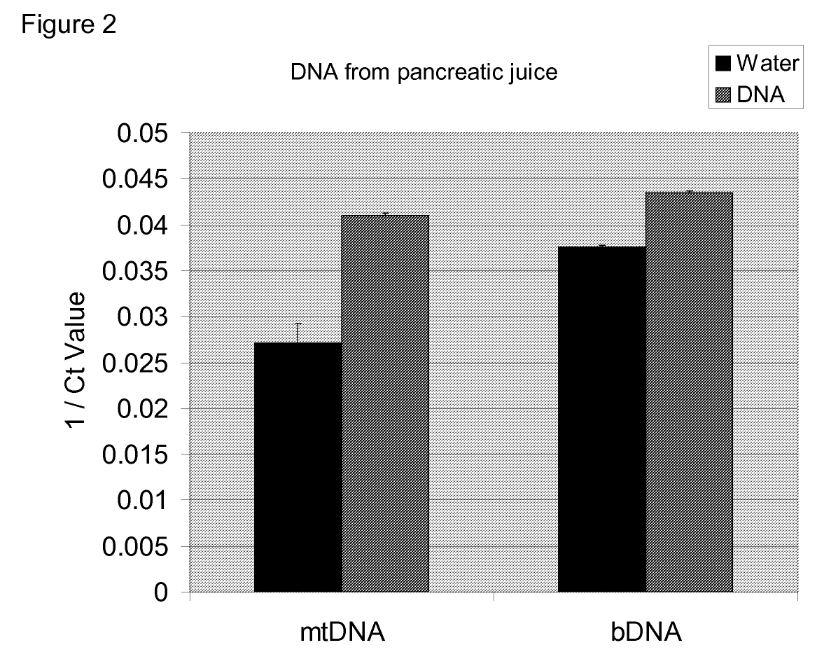

Experiments were performed to determine whether mtDNA was associated with pancreatitis. In these experiments, DNA was isolated from 200 μL pancreatic juice and eluted with 80 μL water. qPCR was performed on the samples using primers for mitochondrial cytochrome B (mtDNA) and 16S rRNA (bDNA). Water was used as a negative control. The results are shown in FIG. 1-3. The data show that mtDNA is detected in pancreatic juice in subjects having pancreatitis (Ct≈24-25) at a level greater than the water control (Ct≈37).

TABLE 1
Exemplary Real Time PCR Primers

| Gene | Sequence | (SEQ ID NO:) |
|---|---|---|
| Human cytochrome B (CytB) | 5′-atgaccccaatacgcaaaat-3′ (forward) | (1) |
| | 5′-cgaagtttcatcatgcggag-3′ (reverse) | (2) |
| Human cytochrome C oxidase | 5′-atgacccaccaatcacatgc-3′ (forward) | (15) |
| subunit III (COX III) | 5′-atcacatggctaggccggag-3′ (reverse) | (16) |
| Human NADH dehydrogenase | 5′-atacccatggccaacctcct-3′ (forward) | (5) |
| (NADH) | 5′-gggcctttgcgtagttgtat-3′ (reverse) | (6) |
| Human GAPDH | 5′-agggccctgacaactctttt-3′ (forward) | (17) |
| | 5′-ttactccttggaggccatgt-3′ (reverse) | (18) |
| Rat cytochrome B | 5′-tccacttcatcctcccattc-3′ (forward) | (7) |
| | 5′-ctgcgtcggagtttaatcct-3′ (reverse) | (8) |
| Rat cytochrome C oxidase | 5′-acataccaaggccaccacac-3′ (forward) | [symbol] ) |
| subunit III | 5′-cagaaaaatccggcaaagaa-3′ (reverse) | (9) |
| Rat NADH dehydrogenase | 5′-caataccccacccccttatc-3′ (forward) | (11) |
| | 5′-gaggctcatcccgatcatag-3′ (reverse) | (12) |
| Rat GAPDH | 5′-gaaatcccctggagctctgt-3′ (forward) | (19) |
| | 5′-ctggcaccagatgaaatgtg-3′ (reverse) | (20) |

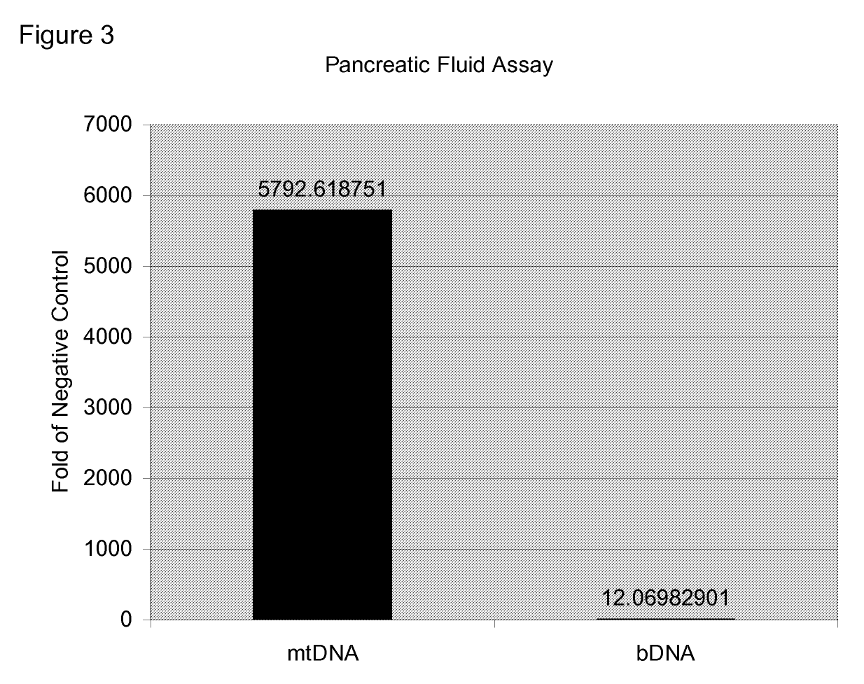

P [symbol] t ~30-35 (data not shown). The level of bacterial DNA detected in the pancreatic fluid samples (Ct ~23), did not differ greatly from water (Ct ~26.5). These data show a significant ratio between the levels of mtDNA and bDNA present in pancreatic fluid from subjects having pancreatitis (~480: 1; FIG. 3). These data indicate an association between organ damage and mtDNA levels. [symbol]
indicates data missing or illegible when filed

### Example 3

### Shiga Toxin-1 Administration Induces an Increase in mtDNA in Baboons

Experiments were performed to determine if a bacterial endotoxin would induce an increase in mtDNA in the serum of baboons. In these experiments, baboons were intravenously administered shiga toxin-1. The levels of mtDNA and bacterial rRNA in the serum of the baboons were measured using qPCR between 0 and 96 hours after shiga toxin-1 administration. The primers used in qPCR were specific for mitochondrial cytochrome B (mtDNA) and bacterial 16S rRNA (bDNA). The primers for baboon mitochondrial cytochrome B (mtDNA) used were: 5′-ATGGAATTTCGGCTCACTTC-3′ (forward primer; SEQ ID NO: 22) and 5′-GAAGGCAGAGGAGGTGTCTG-3′ (reverse primer, SEQ ID NO: 23).

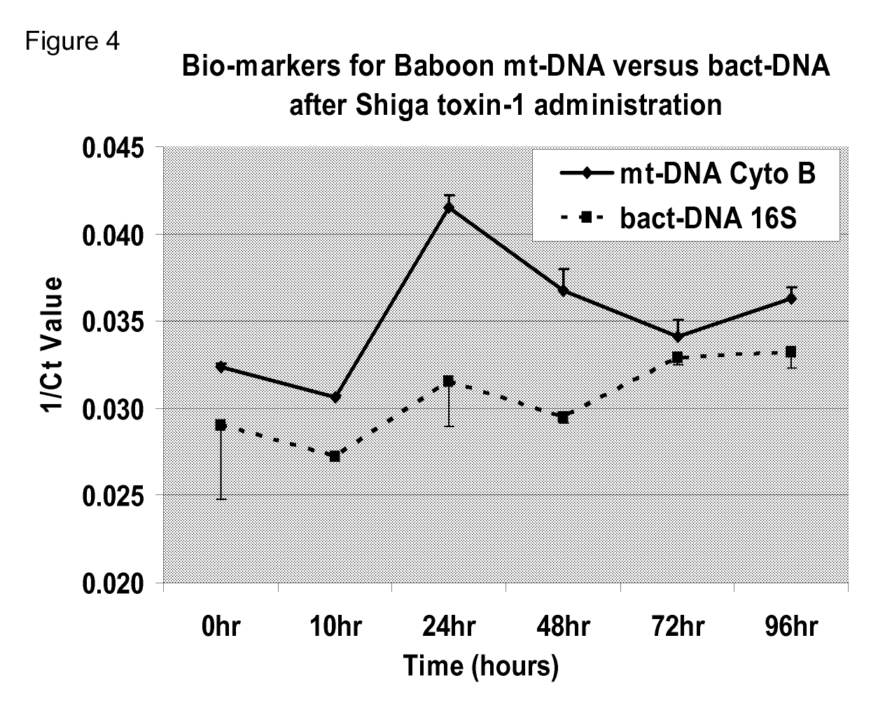

The data demonstrate an increase in the amount of mtDNA levels in the serum of baboons over time following injection with shiga toxin-1 (FIG. 4). The ratio of mtDNA to bacterial DNA also increased over time following injection with shiga toxin-1 (FIG. 4).

### Example 4

### Infection with a Sub-Lethal Dose of E. coli Induces an Increase in mtDNA in Baboons

Experiments were performed to determine whether infection with a sublethal dose of E. coli would induce an increase in mtDNA in the serum of baboons. In these experiments, baboons were administered a sublethal dose of E. coli by 1-hour intravenous infusion. The levels of mtDNA and bacterial rRNA in the serum of the baboons were measured using qPCR between 0 to 48 hours after E. coli infusion. The primers used in qPCR were specific for mitochondrial cytochrome B (mtDNA) and bacterial 16S rRNA (bDNA).

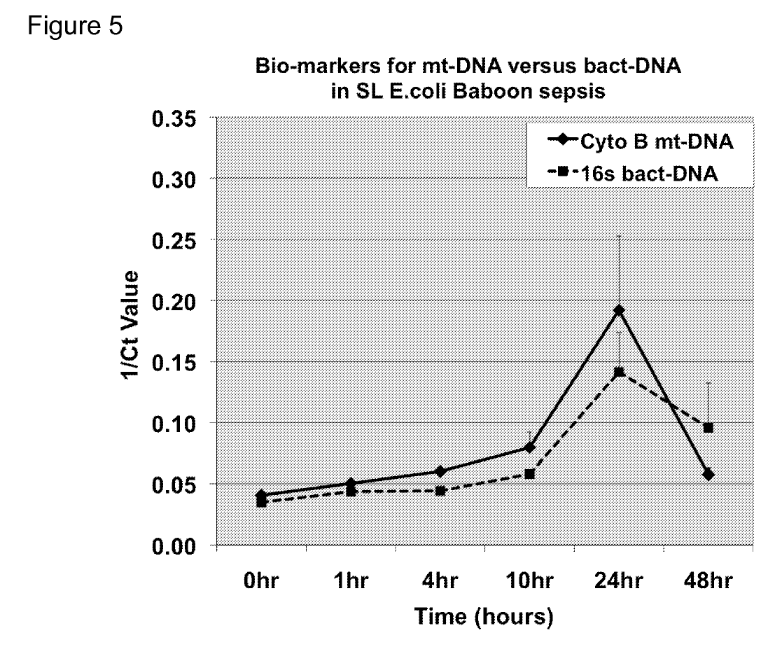

The data demonstrate an increase in serum mtDNA levels over time after E. coli infusion (FIG. 5). The data also show an increase in the ratio of mtDNA to bacterial DNA over time following E. coli infusion (FIG. 5).

### Example 5

### Infection with a Mutant Strain of B. antracis Induces an Increase in mtDNA Prior to Organ Failure

Experiments were performed to determine whether infection with a mutant strain of B. athracis would increase the levels of mtDNA in baboons. In these experiments, baboons received a 1-hour infusion of a dose of a strain of B. anthracis containing a mutation in the gene for anthrax toxin. The B. anthracis strain produces no anthrax toxin. The baboons in these experiments received no further treatment (B. anthracis) or received a dose of activated protein C at the time of B. anthracis infusion (B. anthracis plus APC). Primers for mitochondrial cytochrome B (mtDNA) and bacterial 16S rRNA (bDNA) were used.

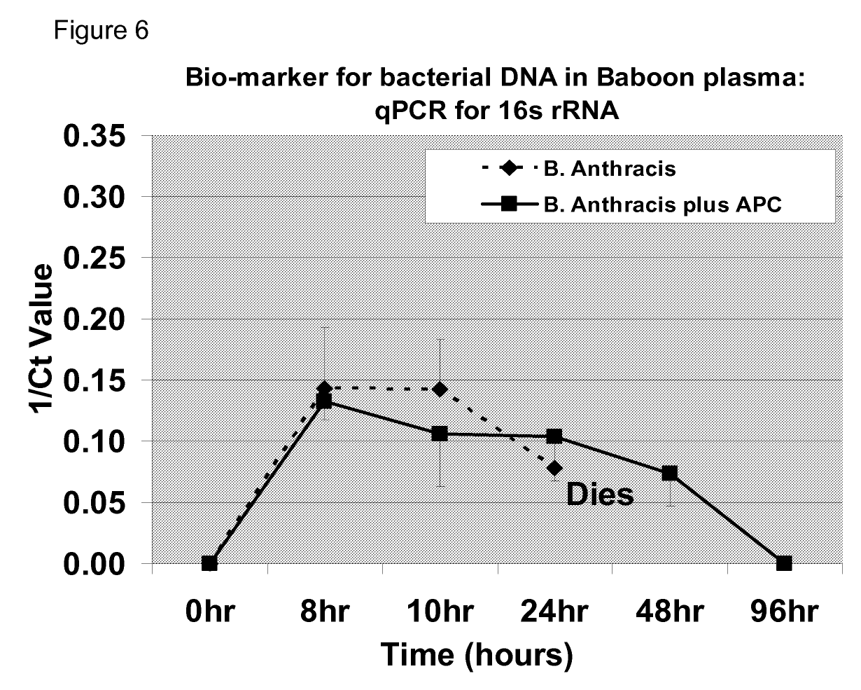

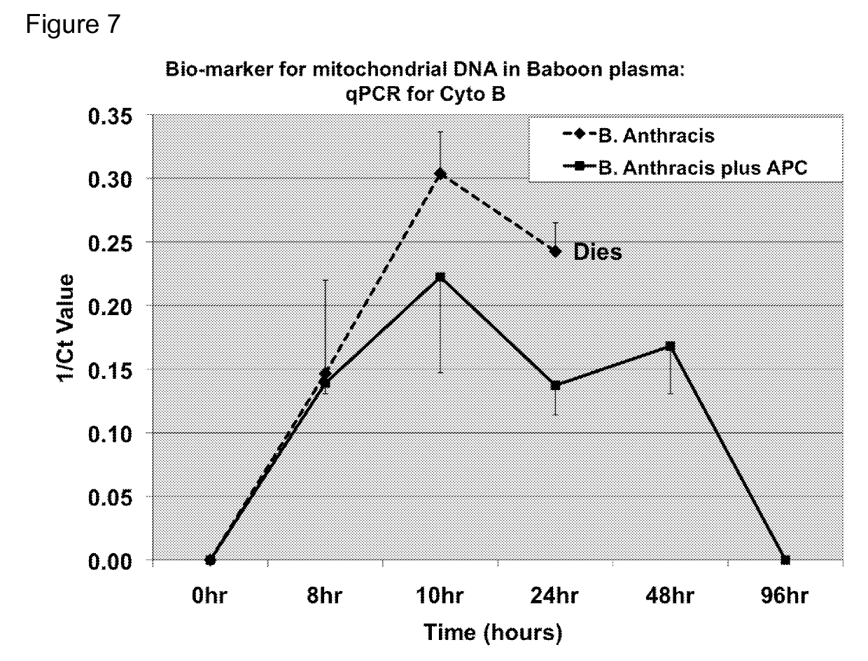

These data show that despite an initial increase in the levels of 16S rRNA levels (bDNA) within the first 8 hours after infection, the level of 16S rRNA stabilizes and later decreases prior to the death of the baboon (FIG. 6). In contrast, the levels of mtDNA increase over the first 10 hours of infection and the increase in more pronounced in baboons receiving no anti-inflammatory treatment (e.g., no APC treatment) (FIG. 7). These data also indicate that the ratio of mtDNA to bacterial nucleic acid (16S rRNA) is increased in baboons prior to the development of organ failure.

### Example 6

### Bacterial and Mitochondrial DNA Assays Distinguish Sepsis from SIRS and Quantify Inflammatory Tissue Injury in Primates

### Abstract

Differentiation between sepsis and SIRS is difficult and, currently, no test assesses tissue injury by sepsis. Plasma mitochondrial DNA (mtDNA) reflects tissue injury and SIRS whereas plasma bacterial 16S DNA (bDNA) might reflect sepsis. Baboons were given 2 hour anthrax infusions with antibiotics, with or without activated protein C (aPC) pre-treatment to block SIRS (n=4 per group). Anthrax infusions caused clear, identical bDNA responses while bacteremia was undetectable by blood culture. bDNA peaked at 10 hours and disappeared by 48 hours in both groups. mtDNA increased, signaling tissue injury with a peak at 24 hours. With aPC rescue, however, mtDNA levels began falling after 24 hours, and the animals survived. Without aPC rescue, mtDNA levels remained elevated, and no animal lived past 96 hours. Control baboons treated with Shiga toxin 1 (ST1) show only tissue injury in the form of increased mtDNA. Sepsis causes tissue injury and SIRS. That SIRS can still be lethal even when all bacteria are killed. Septic PAMPs can be discriminated from endogenous DAMPs using qPCR for mtDNA and bDNA. In primates, sepsis can kill readily through residual SIRS even after sepsis itself has cleared. qPCR for mtDNA and/or bDNA bio-markers can rapidly and accurately define the presence and course of bacteremia, quantify tissue injury incurred by both the septic and sterile mechanisms, and suggest the absence of sepsis.

### Background

The systemic inflammatory response syndrome (SIRS) can occur either in the setting of sepsis due to pathogenic organisms or in a wide variety of circumstances where sterile processes activate inflammation. Both types of “upstream” events signal “danger” to the immune system. In terms of molecular pathogenesis though, infective SIRS (e.g., sepsis) reflects activation of innate immunity by pathogen-associated molecular patterns (PAMPs) whereas sterile SIRS reflects immune activation by ‘damage’ associated molecular patterns (DAMPs) that activate immunity through similar, and in many cases identical, pattern recognition receptors. Conservation of cellular pathways by which DAMPs and PAMPs activate immunity can cause downstream immune response to sepsis and SIRS to be indistinguishable and similarly, the clinical responses to infective and non-infective challenges may be similar. This is important for many reasons, perhaps most imminently so because broad-spectrum antibiotics are often prescribed empirically in SIRS, encouraging the emergence of resistant nosocomial infections.

In current clinical practice, physicians rely upon tests and ‘clinical judgment’ to distinguish between sepsis and SIRS. Although the presence of bacteria at normally sterile sites may be diagnostic of infection, cultures typically take days to grow out and in many cases the bacteria isolated only reflect colonization. Moreover, non-infective and infective inflammation may co-exist. Examples are frequent, but this is commonly seen after trauma, major operation or tissue injury from metabolic processes, like gout.

“Mediators of inflammation” advanced as bio-markers to discriminate between sepsis and SIRS have met with variable success. But since the inflammatory pathways activated by sepsis and SIRS are conserved many downstream mediators produced are also likely to be conserved. We have shown that DNA-based assays for mitochondria can be used to demonstrate tissue injury and broad spectrum bacterial DNA assays can be used to diagnose sepsis. Here we show that qPCR for mitochondrial and bacterial DNA species (mtDNA, bDNA) can be used together to assess the relative contributions of tissue injury and bacterial invasion to SIRS. This facilitates rapid, informed decisions as to whether to treat with or withhold antibiotics or immune modulators.

As is shown below, we assayed serially for bDNA and mtDNA in the plasma of non-human primates (baboons) subjected either to sepsis induced by infusion of an attenuated strain of anthrax or to clinically similar but sterile tissue injury induced by Shiga-toxin 1 (ST1). ST1 is a protein synthesis inhibitor that is the agent of hemolytic-uremic syndrome. The contributions of sepsis and secondary SIRS to organ injury and outcome were further differentiated by treating some baboons with activated protein C (aPC, drotrecogin alfa, activated; Eli Lily and Co., Indianapolis, Ind.) prior to anthrax infusion as a functional knock-out of coagulation and complement induced innate immune responses. In addition to using mtDNA and bDNA as biomarkers for sepsis and SIRS, we show that these markers improve precision in documenting the presence and course of bacteremia as well as quantitating the potential for tissue injury and thus organ failure incurred by septic and sterile insults. Last, show that studying the correlations between DNA markers and organ injury or function help to define the extent to which bacterial PAMPs and endogenous DAMPs contribute to organ dysfunction in clinical sepsis and SIRS.

### Results

Bacterial DNA in Sepsis

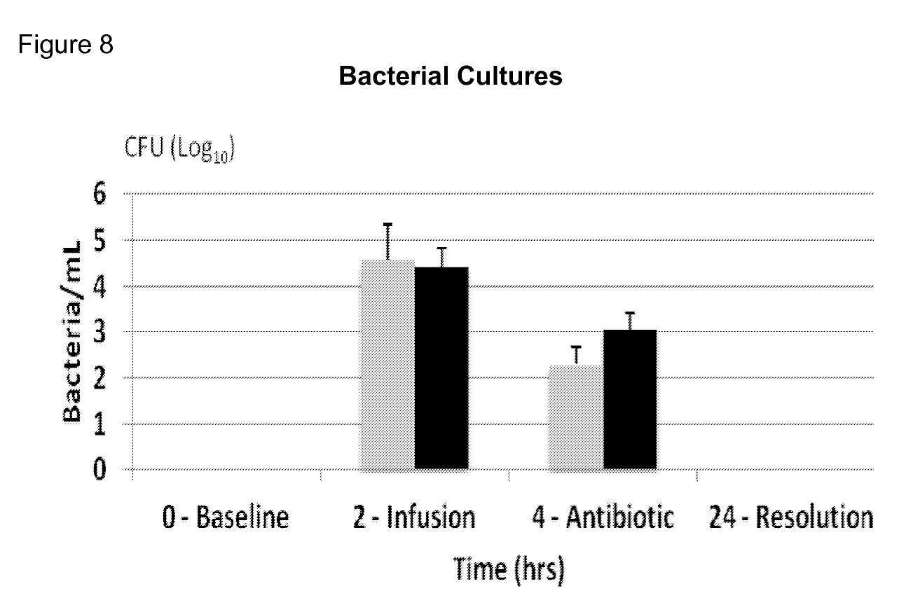

Infusion of anthrax in this primate model resulted in high-grade bacteremia. More than 10⁴ colony-forming units (CFU) of B. anthracis per mL of plasma were found just before the end of the infusion (FIG. 8). Antibiotic administration was then began at 2 hours. Bacterial culture counts fell rapidly thereafter and blood became completely “culture negative” by 24 hours. Pre-treatment with aPC had no effect on blood cultures.

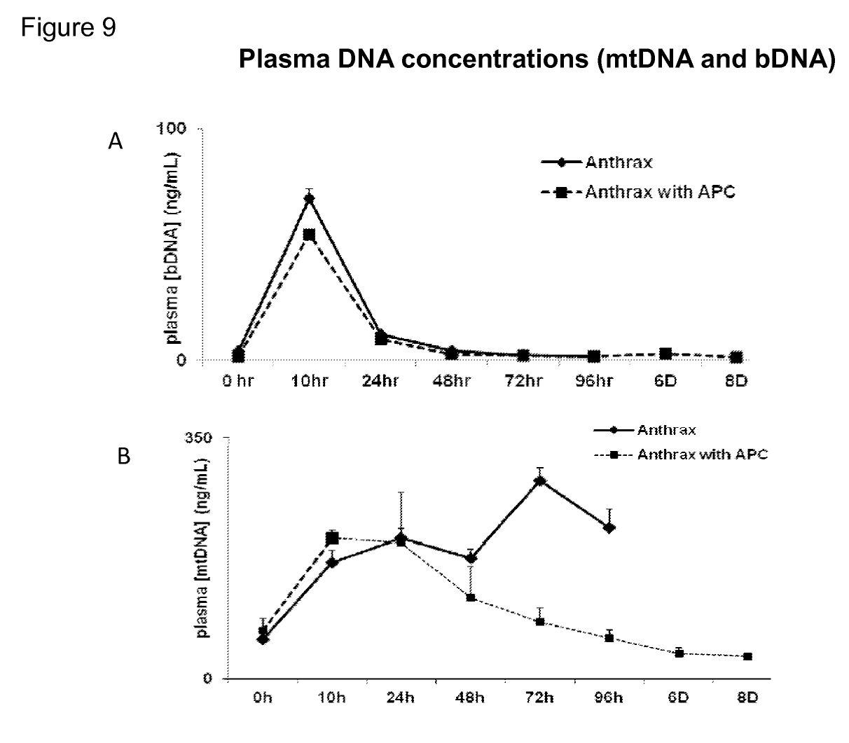

Sepsis was easily recognized using qPCR for 16S bacterial DNA (FIG. 9A) using primers that have no cross reactivity with mitochondrial ribosomal DNA. Peak 16S-bDNA levels were 60-70 ng/mL. As with the quantitative blood cultures, pre-treatment with aPC had no significant effect on peak bDNA load. Also again, bDNA titers fell rapidly. Even though bDNA titers had declined markedly by the end of infusion however, bDNA was still easily detectable at ≧10 ng/mL in plasma at 24 hours (P<0.01 vs basal). Bacterial 16S-rDNA had disappeared from plasma by 48 hours. Baboons treated with aPC (dotted line) demonstrated an essentially identical fall off in 16S-rDNA.

Bacterial and Mitochondrial DNA in Sepsis

mtDNA (CytB) levels rose markedly in parallel with bacteremia in both groups, showing that sepsis caused tissue injury directly and immediately. mtDNA levels during anthrax bacteremia peaked at approximately 200 ng/mL either with or without aPC pretreatment (FIG. 9B). In aPC untreated animals however, mtDNA levels remained persistently elevated (solid line) at 48 hours and beyond. This reflected ongoing tissue injury well after the disappearance of both bacteria and bDNA from the blood, and all these animals died. In distinction, baboons pre-treated with aPC (dotted line) suffered near identical initial tissue injury due to sepsis but subsequently however, their plasma mtDNA levels decayed. This reflected a marked attenuation of tissue injury after resolution of bacteremia and all these animals survived. Noting that the two groups had cleared their bacteremias in an identical fashion (FIGS. 8, 9A) we conclude that the resolution of post-bacteremic tissue injury seen reflected attenuation of the SIRS response by aPC.

Bacterial and Mitochondrial DNA in Sterile Tissue Injury

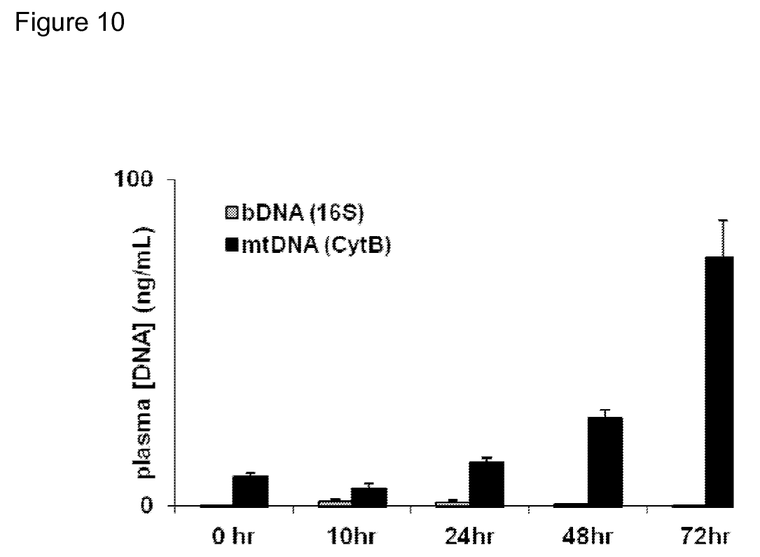

Shiga Toxin 1 (ST1) inhibits protein synthesis causing cellular injury. ST1 is also the agent of Hemolytic-Uremic Syndrome(HUS) after E. Coli O157:H7. In the model we use, it causes progressive cellular injury, organ dysfunction and death. ST1 is itself sterile however, and its administration therefore causes a toxic rather than an infective injury and we used ST1 administration to evaluate mtDNA release after a completely sterile tissue injury. The results show that subsequent to ST1 administration (FIG. 10) mtDNA appeared in plasma at progressively increasing concentrations. Thus cell injury was progressive until day 3, after which point all animals required euthanasia. In contrast, baboons intoxicated with ST1 showed insignificant levels of circulating bDNA (FIG. 10) confirming that cellular injury and death after ST1 were independent of bacteremia, either from exogenous infection or indirectly from an endogenous sources like “gut translocation”. A progressive tissue injury signal is seen in the form of mtDNA. There is no PCR evidence of bacterial infection. The ratio of mtDNA/bDNA increases geometrically with progression of illness.

Bacterial and Mitochondrial DNA in Non-Lethal Infections

To model infection without the induction of a SIRS response, we elected to use a non-lethal E. coli bacteremia. In this case (FIG. 5) we see that bacterial 16S-DNA becomes markedly elevated during the infusion phase but its concentration decays rapidly as the circulating bacteria are subsequently cleared by the host. As with lethal anthrax sepsis, mtDNA concentration parallels 16s-bDNA during the period of bacteremia. Critically though, the injury signal (CytB mtDNA) disappears immediately after the cessation of infusion, returning to baseline values even before bacteremia is totally cleared. This contrasts strongly with the events in lethal Anthrax sepsis. There, the tissue injury signal persists long after clearance of the bacteremia and the animals die (FIG. 9B). These latter events strongly suggest that a self-perpetuating SIRS response due to release of cellular ‘danger’ signals [or ‘alarmins’] (which include mtDNA) is initiated by lethal, but not by non-lethal bacteremia. This finding also shows that mtDNA may be an appropriate “biomarker” for the severity and outcome of sterile SIRS initiated by septic tissue injury.

Multiple Organ Failure in SIRS versus Sepsis

Cardiorespiratory Dysfunction

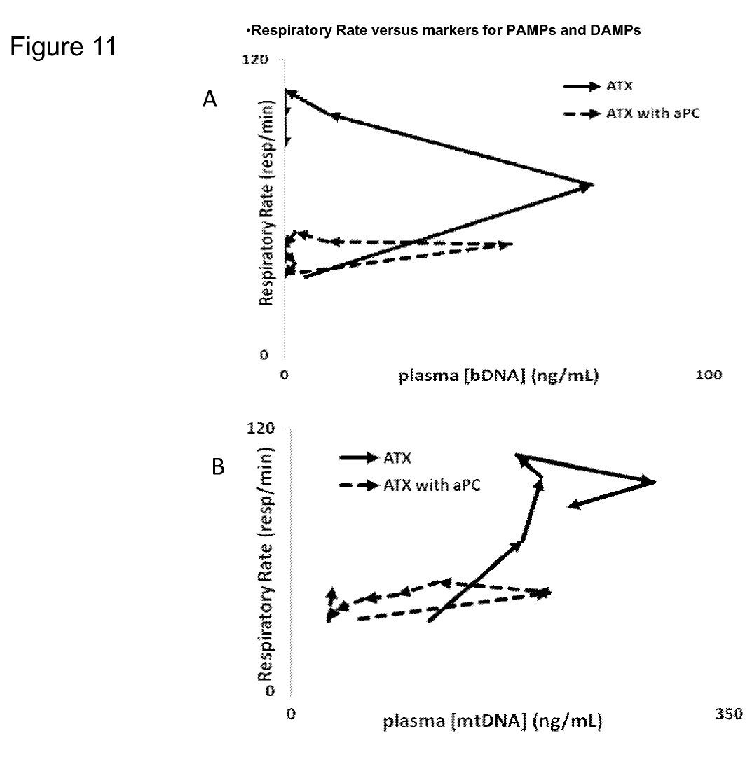

Heart rate (HR) and respiratory rate (RR) were studied as clinical markers for the effects of sepsis on the heart and lungs. We saw that both HR and RR rose immediately with the onset of bacteremia (FIG. 11). We found that changes in RR over time were closely related to mtDNA concentration (FIG. 11B) rather than to the concentration of 16S-bDNA (FIG. 11A). With aPC administration RR remained normal irrespective of both the DAMP and PAMP concentrations. Thus aPC altered the relationship between inflammatory stimuli and respiratory rate. Respiratory rate did not vary with bDNA (FIG. 11A), rather, without aPC respiratory rate simply increased over time. In all cases aPC prevented tachypnea. This suggests that the tissue injury from sepsis rather than the septic event itself drives tachypnea, even after septic stimuli are cleared. Thus it appears that where sepsis damages tissue, persistent circulation of DAMPs drives the respiratory response to sepsis long after PAMPs have been cleared.

Hepatic and Renal Failure

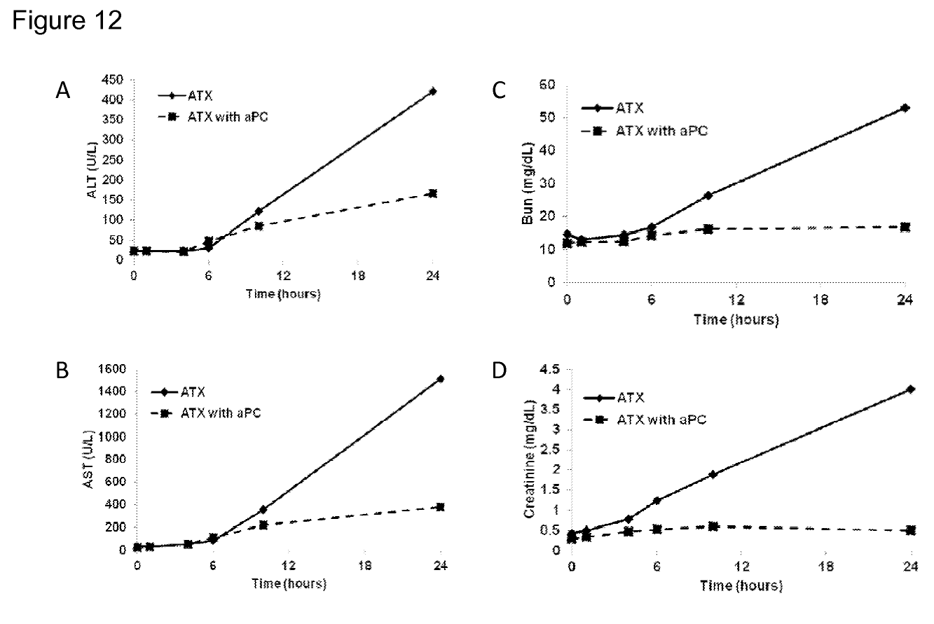

Plasma transaminases (AST, ALT) were studied as markers for hepatocellular injury in sepsis and SIRS over the first 24 hours (FIGS. 12A and 12B). Bilirubin showed a similar pattern. Blood urea nitrogen (BUN) and creatinine were studied similarly as markers for kidney injury (FIG. 12C and 12D). All these markers of septic solid organ injury showed dramatic increases after anthrax infusion. Increases began at around 6 hours and continued through 24 hours. Thus liver and kidney function were progressively compromised in sepsis due to b. anthracis.

Liver and kidney function (FIG. 12) were markedly spared in septic animals pretreated with aPC. Lesser effects on organ function occurred despite the cohorts clearing bacteria and bDNA identically. This suggests that inflammatory responses to sepsis cause the preponderance of cellular injury in anthrax sepsis rather than the presence of bacteria themselves. Accordingly, decreased organ dysfunction after aPC treatment was directly correlated with lesser evidence of cellular injury as manifested by circulating mtDNA.

Hematologic Failure

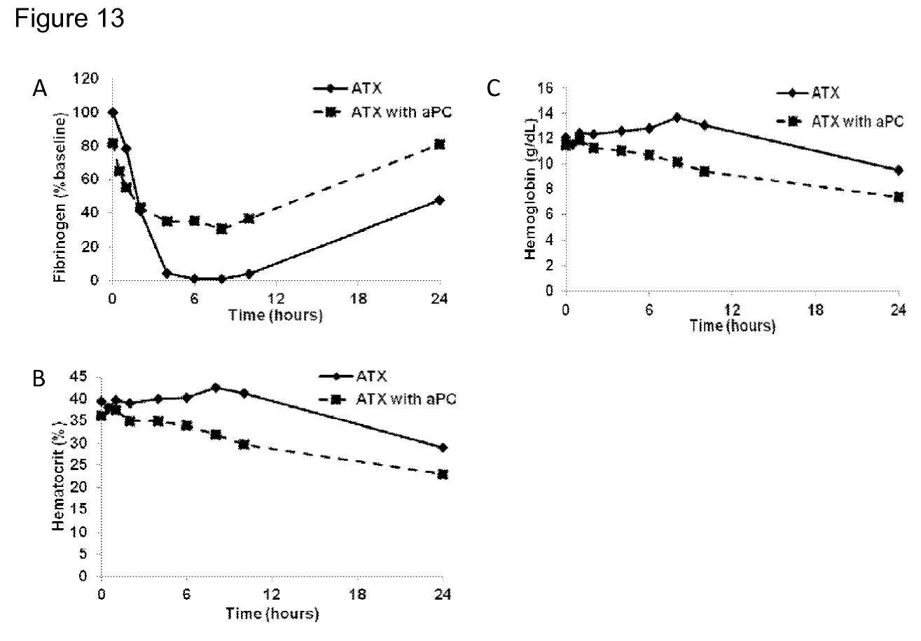

Plasma fibrinogen (FIG. 13A) and platelet levels can fall early in high-grade sepsis complicated by the disseminated intravascular coagulation (DIC) syndrome. Fibrinogen can also then rebound subsequently as a reflection of the hepatic acute-phase response. Hematocrit and hemoglobin rose early in sepsis reflecting capillary leak syndrome and hemo-concentration (FIG. 13B and 13C). We saw here that anthrax sepsis led to a rapid and precipitous fall in fibrinogen (and in platelets) during the period of bacteremia itself. This was followed by slow recovery. Decreases in fibrinogen (and in platelets) were not mitigated by aPC. This suggests DIC may be a direct effect of sepsis rather than being the result of SIRS.

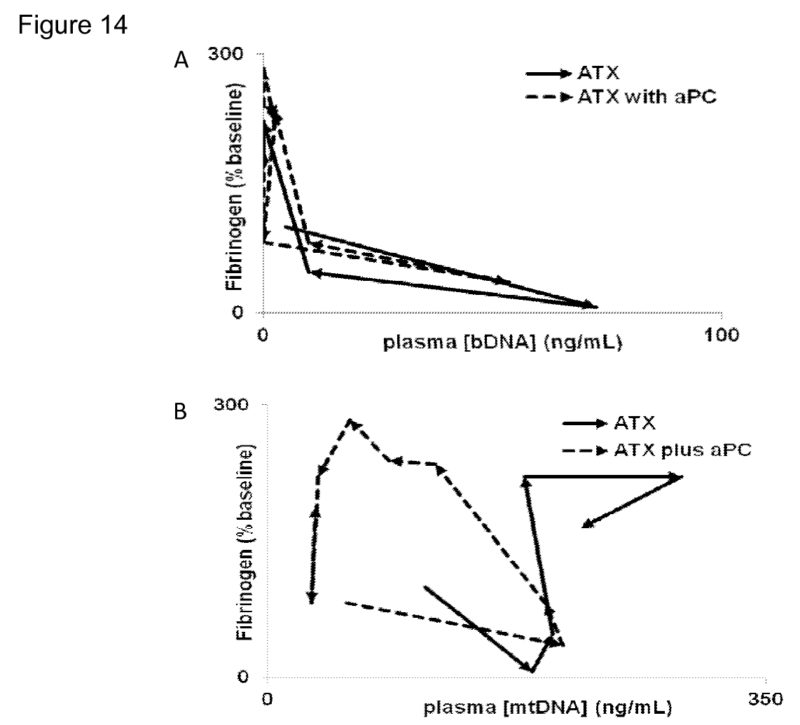

We see fibrinogen as a function of tissue injury, represented by mtDNA (FIG. 14A), and we also see fibrinogen as a function of bacteremia, as represented by bDNA (FIG. 14B). Regardless of treatment mtDNA concentration does not correlate to plasma fibrinogen level (FIG. 14A). However, DIC (as indicated by defibrination), appears to be strongly linked to bacteremia (as indicated by bDNA) (FIG. 14B). Thus, the presence of bacterial PAMPs may be more specific for the induction of DIC where tissue injury and the sterile inflammatory responses produced by it do not cause DIC, even when initiated by bacteremia.

### Discussion

Sepsis often kills the host through residual SIRS even after sepsis itself has cleared. PAMPs and DAMPs are the proximal initiators of SIRS responses to sepsis and tissue injury respectively, but their overlapping effects often make it difficult to determine whether patients are manifesting continuing sepsis, SIRS due to sterile injury, or SIRS due to a prior episode of sepsis. We have studied this critical clinical issue using a highly relevant, non-human primate model of treated but lethal bacteremia and related models of sublethal sepsis and pure sterile tissue injury. Bacterial 16S-rDNA was used as a biomarker for sepsis but not tissue injury, and mitochondrial Cytochrome B DNA was used as a biomarker for cellular injury and resultant SIRS independent of bacteremia. Methodologic control studies showed that our detection threshold for 16S-bDNA was about 100 fg/mL without cross-recognition of mtDNA. This level of sensitivity and specificity suggests such assays lend themselves to clinical relevant discrimination between sepsis and SIRS.

The data show for the first time that SIRS due to sepsis can be resolved into an initial period of direct tissue injury and a later period of sterile inflammation. The progressive release of mitochondrial DNA seen in sepsis, but not the initial septic injury, was reproduced when sterile tissue injury was initiated directly by Shiga Toxin. This progressive, secondary tissue injury perpetuated release of tissue-derived DAMPs like mtDNA and was suppressed by anti-inflammatory therapy. Thus the data indicate that sepsis can cause SIRS and organ dysfunction directly and also show how it can cause SIRS secondarily when tissue injury releases DAMPs that activate innate immunity and can cause organ dysfunction.

The proximate cause of morbidity and mortality after sepsis and SIRS is usually inflammatory multiple organ dysfunction (MOF). We explored the roles of pathogen derived motifs and endogenous ‘damage’ motifs in end-organ dysfunction by seeking significant relationships between their concentrations and the evolution of septic MOF with and without aPC pretreatment. We had noted (FIGS. 8 and 9A) that aPC had no effect on the concentration of PAMPs per se and that preventing late cellular injury and release of DAMPs was associated with prevention of organ failure.

Preventing tissue DAMP release showed that the syndromes of pure sepsis and sepsis-followed-by SIRS overlapped but were not identical. Defining ‘septic’ responses as those driven by bacteremia alone and ‘septic SIRS’ as being driven by bacteremia plus the release of DAMPs, we see significant variance between the two syndromes. Using Pearson correlation coefficients, we found several noteworthy associations. First, we used respiratory rate (RR) as a simple assessment of acute lung injury (ALI). RR is a complex variable later as the animals become acidotic and develop respiratory failure. But we believe that until then, tachypnea is generally a good marker for early ALI. So we saw here that aPC fundamentally altered the relationship between sepsis and RR. Respiratory rate did not correlate significantly with plasma bDNA (9a). Without aPC RR simply increased over time until terminal decompensation. After aPC pre-treatment, RR was unchanged even as bDNA and mtDNA rose and fell during the acute bacteremia. In distinction RR varied directly and significantly with mtDNA concentration in untreated sepsis (P<0.01, FIG. 11A solid line). After aPC pre-treatment however, RR failed to vary with mtDNA (P<0.01). These findings suggest that DAMPs from tissue injury (rather than PAMPs) drive tachypnea in sepsis, and that aPC acted to block pathways linking DAMPs to tachypnea after septic stimuli were cleared.

A very different picture was seen examining the hematologic manifestations of sepsis and SIRS. We can see fibrinogen level over time represented as a function of tissue injury (mtDNA, FIG. 14A) and we also see it represented as a function of bacteremia (bDNA, FIG. 14B). In no case did plasma fibrinogen level bear any relationship to mtDNA (FIG. 14A). On the contrary, fibrinogen concentration was strongly inversely linked to bacteremia (P<0.01, FIG. 14B). Thus bacterial PAMPs appear to be associated specifically with defibrination whereas tissue injury per se did not cause DIC, even when it was initiated by bacteremia. Thus also, although less sensitive than bDNA as a biomarker, defibrination may prove a very specific predictor of bacteremia.

Antibiotics can kill bacteria but cannot eliminate SIRS. Biologic response modifiers like aPC may block SIRS by preventing a vicious cycle of inflammation, cellular injury and release of DAMPs. But suppression of inflammation is achieved at the risk of potentiating persistent infections. Thus although antibiotics and anti-inflammatory therapies are potentially complementary, combining them is most likely to achieve improved outcomes if they are used at a time and in a sequence appropriate to ongoing molecular pathophysiology. We propose that biomarkers such as those studied here provide direction in the timing and use of antibiotics and biologic response modifiers in clinical practice.

### Materials and Methods

Ethical Considerations

Animal studies were performed under the oversight of the Institutional Animal Care and Use Committee of University of Oklahoma Health Sciences Center as well as of Boston University Medical Center and Beth Israel Deaconess Medical Center where appropriate. All studies were performed in strict compliance with applicable National Institutes of Health guidelines.

Baboons

All the primate experiments were performed at Oklahoma Health Sciences Center. Papio c. cynocephalus or Papio c. Anubis were purchased and cared for as appropriate. Bacillus anthracis, Sterne strain (ATX) was prepared for infusion. Because toxins are known virulence factors for B. anthracis, and recombinant activated protein C is known to influence only septic responses, we evaluated the influence of aPC in baboons challenged with an unencapsulated strain (Delta Sterne) that has been altered to remove the plasmid that encodes the exotoxin.

On Day 0, Papio c. cynocephalus baboons (5-7 kg) were anesthetized, intubated and catheterized for i.v. infusions. Half of the baboons (4) were randomized to receive pre-treatment with activated protein C (aPC). Animals treated with aPC received a bolus injection of 3 mg/kg at T (−10min). A two-hour bacterial infusion was initiated at T₀ hrs at 0.7-3⁹ CFU/kg. Infusion of aPC at 64 ug/kg/min was continued for 6 hours. All animals received a dose of levofloxacin (7 mg/kg) four hours after the start of the bacterial infusion and daily thereafter. The study endpoint was set at 7 days to monitor disease progression. All 7 day survivors were visibly recovered and had no clinical appearance of illness.

Bacteremia was confirmed by traditional plating methods using blood obtained at T=2 hours just after finishing the infusion and at T=4 hours, just before the antibiotics were given. Colony counts varied according to the loading dose. For a 1E8 CFU/kg challenge, colony counts were near 1E4 CFU/ml at T=2 hours and 100 CFU/ml at T=4 hours. Colony counts on blood sampled between days 2 to 7 were consistently negative. Complete blood counts, blood chemistries, renal and liver function tests and fibrinogen were determined at the times noted below. Vital signs including temperature (T), respiratory rate (RR) and heart rate (HR) were monitored by polygraph.

Sample Processing

Blood was drawn at the time points indicated from baboons. In all cases, plasma was collected by centrifuging whole blood for 10 minutes at 200×g and then transferred to a new tube. Plasma used for PCRs was then spun twice at 3000×g to remove residual cells, platelets, micro-particles and other debris.

DNA Isolation

Details of DNA isolation can be found in QIAamp DNA Blood Mini Kit manual. DNA was prepared from 100 μL plasma using QIAamp DNA Blood Mini Kit from Qiagen, according to the manufacturer's protocol, except that 80 μL was used to elute DNA from spin column.

Real-Time PCR Protocols

The same amount of DNA (5 μL) was used for each real-time PCR reaction using SYBR Green Master Mix (Applied Biosystems) by Mastercycler EP Realplex (Eppendorf), StepOne Plus (Applied Biosystems), or Mx3000P (Agilent Technologies).

CytB qPCR should be accompanied with a standard curve of mtDNA from the same species as the sample. Primers target species-specific CytB DNA that have also been shown not to cross-react with bacterial 16S DNA.

Cytochrome-B (CytB) primers were chosen for study because unique among mitochondrial molecules, CytB is essentially absent from bacteria on BLAST study. Similarly, PCRs targeting 16S bacterial ribosomal RNA have long been used as broad spectrum probes for bacteria. We noted however, that 12s mitochondrial RNA bears many similarities to bacterial 16S-RNA; creating the possibility of false positive assays. We therefore picked 16S bDNA targets that were evolutionarily distant from mtDNA ribosomal sequences.

Since we can see variation from plate to plate due to manufacturing differences and variation between PCR machines or using different batches of reagents, we elected to create standard curves on each plate to quantify DNA concentration using either commercial E. coli DNA (Invivogen) or mtDNA purified from freshly prepared mitochondria using the Wako mtDNA Extractor CT kit. qPCR assays demonstrate the high sensitivity and specificity of our assays for human mtDNA and authentic bDNA from Gram-positive (Staph. aureus), Gram-negative (E. coli) and anaerobic (Bacteroides fragilis) organisms. In all cases there is essentially no cross reactivity although the primers are sensitive down to femtogram/mL levels.

Data Analysis

Statistics. Data were analyzed for differences between baboon groups using Student's T-test, assuming equal variance. To identify any predictive potential of plasma mtDNA or bDNA in the anthrax baboon model, Pearson Correlation coefficients were calculated using SPSS between mtDNA and bDNA levels and the clinical measurements. Relationships found to have a P<0.01 on initial analysis were subjected to further analysis.

### Example 7

Patient N.O. is a 35 year old woman with known sickle cell disease. She presented with fever, jaundice and abdominal pain. The WBC count on day 1 was >25,000. CT of the abdomen showed both dilation of the biliary ducts and splenic auto-infarction. Thus, her presentation could have been due 1) to sepsis secondary to common duct stones and cholangitis, or 2) to sterile splenic infarction. The patient underwent endoscopic retrograde cholangiopancreatography (ERCP) on day 2, which found no cholangitis. The WBC count went to >40,000, the patient developed multiple organ failure and required mechanical ventilation. Blood cultures were sterile on five occasions over the time period. Bronchoalveolar lavages looking for pneumonia (or a new pneumonia) were sterile on 2 occasions. The patient had 2 changes in her antibiotics based on the assumption of sepsis refractory to current management. She underwent splenectomy on day 5 with subsequent slow resolution of her illness.

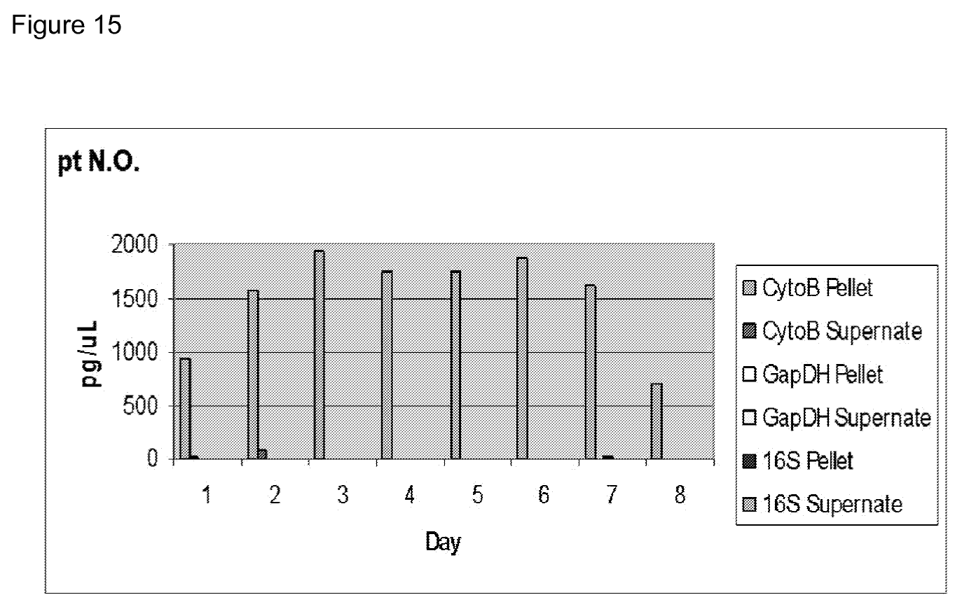

FIG. 15 shows patient N.O.'s plasma levels of mitochondrial Cyto-B DNA (blue) and bacterial 16S-DNA (red) over time. The ratio of mitochondrial to bacterial DNA was generally in the range of 100:1 to 1000:1. If used clinically, these data would have strongly supported that patient N.O.'s illness reflected a sterile SIRS response to dead tissue rather than that infective sepsis from invasive infection.

This data would support three key changes in therapy:

1) The absence of a need for an ERCP (with its attendant risks);

2) the absence of a need for antibiotics; and

3) An earlier splenectomy with potential avoidance of the episode of organ failure and mechanical ventilation.

### OTHER EMBODIMENTS

From the foregoing description, it will be apparent that variations and modifications may be made to the invention described herein to adopt it to various usages and conditions. Such embodiments are also within the scope of the following claims.

All publications, patent applications, and patents mentioned in this specification are herein incorporated by reference to the same extent as if each independent publication, patent application, or patent was specifically and individually indicated to be incorporated by reference. In particular, U.S. Ser. No. 61/419,502 in hereby incorporated by reference in its entirety.

From the foregoing description, one skilled in the art can easily ascertain the essential characteristics of this invention; can make various changes and modifications of the invention to adapt it to various usages and conditions. Thus, other embodiments are also within the claims.

What is claimed is:

1. A method for diagnosing or determining the likelihood that a subject will develop one or more infection-induced illness(es) comprising the steps of:
a) measuring the amount of bacterial nucleic acid or peptide in at least one sample from said subject;
b) measuring the amount of a mitochondrial nucleic acid or peptide in said at least one sample or in a second sample from said subject; and
c) comparing the amount of bacterial nucleic acid or peptide measured in step (a) with the amount of mitochondrial nucleic acid or peptide measured in step (b),
wherein an increased ratio of the amount of mitochondrial nucleic acid or peptide to the amount of bacterial nucleic acid or peptide indicates the subject has an increased likelihood of later developing one or more infection-induced illness(es), the subject has one or more infection-induced illness(es), or the subject is likely to develop one or more severe infection-induced illness(es) in the future.

2. The method of claim 1, wherein said one or more infection-induced illness(es) are selected from the group consisting of: organ failure, hypotension, seizures, shock, increased heart rate, tachypnea, decreased arterial pressure of CO₂, and hemolytic-uremic syndrome.

3. (canceled)

4. The method of claim 1, wherein said subject has a bacterial infection.

5. The method of claim 4, wherein the bacterial infection is a local infection.

6. The method of claim 4, wherein the bacterial infection is a systemic infection.

7. The method of claim 1, wherein said subject does not demonstrate any symptoms of severe septic shock.

8. The method of claim 4, wherein the bacterial infection is caused by one or more bacteria selected from the group consisting of: Bacillus athracis, Vibrio cholera, Bordetella pertussis, Eschericia coli, Clostridium tetani, Clostridium perfringes, Clostridium difficile, Clostridium botulinum, Listeria monocytogenes, Streptococcus spp., Staphylococcus aureus, Mycobacterium tuberculosis, Corynebacterium diphtheria, Shigella dysenteriae, Pseudomonas aeriginosa, and Bacillus thuringiensis.

9. (canceled)

10. The method of claim 1, wherein said at least one sample or said second sample is obtained from a subject within 24 hours of an initial presentation of the subject to a medical professional.

11. The method of claim 1, wherein said at least one sample or said second sample is obtained from the subject at least 24 hours after an initial presentation of the subject to a medical professional.

12.-15. (canceled)

16. The method of claim 1, wherein said at least one sample or said second sample is obtained from said subject within 3 to 24 hours after a potential exposure to an endotoxic bacterium or a composition containing an endotoxin.

17. The method of claim 1, wherein the mitochondrial nucleic acid encodes cytochrome B, cytochrome C oxidase subunit III, or NADH dehydrogenase.

18.-53. (canceled)

54. A method of treating a subject with a bacterial infection comprising diagnosing or determining the likelihood that a subject will develop one or more infection-induced illness(es) according to the method of claim 1 and administering to said subject having an increased ratio of the amount of mitochondrial nucleic acid or peptide to the amount of bacterial nucleic acid or peptide one or more anti-inflammatory agents and one or more antimicrobial agents.

55. The method of claim 54, wherein the one or more anti-inflammatory agents are selected from anti-FPR1 antibodies, cyclosporine H, activated protein C, chloroquine, or anti-TLR9 antibodies.

56. The method of claim 54, wherein said subject is administered one or more doses of one or more antimicrobial agents prior to the administration of one or more doses of one or more anti-inflammatory agents.

57. The method of claim 56, wherein the administration of the one or more anti-inflammatory agents is administered at least 12 hours after the administration of the one or more antimicrobial agents.

58. The method of claim 54, wherein said treatment reduces the likelihood of developing one or more infection-induced illness(es) or reduces the likelihood of death resulting from one or more infection-induced illness(es).

59. The method of claim 58, wherein said infection-induced illness(es) are selected from the group consisting of: organ failure, hypotension, seizures, shock, increased heart rate, tachypnea, decreased arterial pressure of CO₂, and hemolytic-uremic syndrome.

60.-74. (canceled)

75. A kit for diagnosing or determining the likelihood that a subject will develop one or more infection-induced illness(es) comprising:
a) one or more first oligonucleotide primers effective for the amplification of a bacterial nucleic acid or one or more antibodies that specifically bind to one or more bacterial peptides;
b) one or more second oligonucleotide primers effective for the amplification of a mitochondrial nucleic acid or one or more antibodies that specifically bind to one or more mitochondrial peptides; and
c) instructions for using said first and second oligonucleotide primers or said antibodies for determining the likelihood the subject will develop one or more infection-induced illness(es), for diagnosing the subject as having one or more infection-induced illness(es), or for predicting the future severity of one or more infection-induced illness(es) in the subject.

76.-87. (canceled)

88. The method according to claim 1, wherein said bacterial nucleic acid is 16S ribosomal RNA, and wherein said mitochondrial nucleic acid encodes cytochrome B, cytochrome C oxidase subunit III, Glyceraldehyde 3-phosphate dehydrogenase, or NADH dehydrogenase.

89. The method according to claim 1, wherein said mitochondrial peptide is NADH dehydrogenase subunit I, NADH dehydrogenase subunit II, NADH dehydrogenase subunit III, NADH dehydrogenase subunit IV, NADH-ubiquinone oxidoreductase chain 4L, NADH dehydrogenase subunit V, NADH dehydrogenase subunit VI, cytochrome B, cytochrome C oxidase subunit I, cytochrome C oxidase subunit II, cytochrome C oxidase subunit III, ATP synthase F0 subunit VI, ATP synthase subunit VIII, or Glyceraldehyde 3-phosphate dehydrogenase.


SEQUENCE LISTING (21 sequences)

SEQ ID NO: 1 - DNA, 20 bp, Artificial Sequence
  Forward primer for human cytochrome B
atgaccccaatacgcaaaat

SEQ ID NO: 2 - DNA, 20 bp, Artificial Sequence
  Reverse primer for human cytochrome B
cgaagtttcatcatgcggag

SEQ ID NO: 3-4 - skipped (2 entries)

SEQ ID NO: 5 - DNA, 20 bp, Artificial Sequence
  Forward primer for human NADH dehydrogenase
atacccatggccaacctcct

SEQ ID NO: 6 - DNA, 20 bp, Artificial Sequence
  Reverse primer for human NADH dehydrogenase
gggcctttgcgtagttgtat

SEQ ID NO: 7 - DNA, 20 bp, Artificial Sequence
  Forward primer for rat cytochrome B
tccacttcatcctcccattc

SEQ ID NO: 8 - DNA, 20 bp, Artificial Sequence
  Reverse primer for rat cytochrome B
ctgcgtcggagtttaatcct

SEQ ID NO: 9 - DNA, 19 bp, Artificial Sequence
  Forward primer for rat cytochrome C oxidase subunit III
acataccaaggccaccaac

SEQ ID NO: 10 - DNA, 20 bp, Artificial Sequence
  Reverse primer for rat cytochrome C oxidase subunit III
cagaaaaatccggcaaagaa

SEQ ID NO: 11 - DNA, 20 bp, Artificial Sequence
  Forward primer for rat NADH dehydrogenase
caataccccacccccttatc

SEQ ID NO: 12 - DNA, 20 bp, Artificial Sequence
  Reverse primer for rat NADH dehydrogenase
gaggctcatcccgatcatag

SEQ ID NO: 13 - DNA, 21 bp, Artificial Sequence
  Forward primer for bacterial 16S ribosomal RNA
tgtagcggtgaaatgcgtaga

SEQ ID NO: 14 - DNA, 22 bp, Artificial Sequence
  Reverse primer for bacterial 16S ribosomal RNA
ccagggtatctaatcctgtttg

SEQ ID NO: 15 - DNA, 20 bp, Artificial Sequence
  Forward primer for human cytochrome C oxidase subunit III
atgacccaccaatcacatgc

SEQ ID NO: 16 - DNA, 20 bp, Artificial Sequence
  Reverse primer for human cytochrome C oxidase subunit III
atcacatggctaggccggag

SEQ ID NO: 17 - DNA, 20 bp, Artificial Sequence
  Forward primer for human GAPDH
agggccctgacaactctttt

SEQ ID NO: 18 - DNA, 20 bp, Artificial Sequence
  Reverse primer for human GAPDH
ttactccttggaggccatgt

SEQ ID NO: 19 - DNA, 20 bp, Artificial Sequence
  Forward primer for rat GAPDH
gaaatcccctggagctctgt

SEQ ID NO: 20 - DNA, 20 bp, Artificial Sequence
  Reverse primer for rat GAPDH
ctggcaccagatgaaatgtg

SEQ ID NO: 21 - DNA, 16571 bp, Homo sapiens
gatcacaggtctatcaccctattaaccactcacgggagctctccatgcatttggtattttcgtctggggggtgtgcacgcgatagcattgcgagacgctggagccggagcaccctatgtcgcagtatctgtctttgattcctgcctcattctattatttatcgcacctacgttcaatattacaggcgaacatacctactaaagtgtgttaattaattaatgcttgtaggacataataataacaattgaatgtctgcacagccgctttccacacagacatcataacaaaaaatttccaccaaacccccccctccccccgcttctggccacagcacttaaacacatctctgccaaaccccaaaaacaaagaaccctaacaccagcctaaccagatttcaaattttatctttaggcggtatgcacttttaacagtcaccccccaactaacacattattttcccctcccactcccatactactaatctcatcaatacaacccccgcccatcctacccagcacacacacaccgctgctaaccccataccccgaaccaaccaaaccccaaagacaccccccacagtttatgtagcttacctcctcaaagcaatacactgaaaatgtttagacgggctcacatcaccccataaacaaataggtttggtcctagcctttctattagctcttagtaagattacacatgcaagcatccccgttccagtgagttcaccctctaaatcaccacgatcaaaagggacaagcatcaagcacgcagcaatgcagctcaaaacgcttagcctagccacacccccacgggaaacagcagtgattaacctttagcaataaacgaaagtttaactaagctatactaaccccagggttggtcaatttcgtgccagccaccgcggtcacacgattaacccaagtcaatagaagccggcgtaaagagtgttttagatcaccccctccccaataaagctaaaactcacctgagttgtaaaaaactccagttgacacaaaatagactacgaaagtggctttaacatatctgaacacacaatagctaagacccaaactgggattagataccccactatgcttagccctaaacctcaacagttaaatcaacaaaactgctcgccagaacactacgagccacagcttaaaactcaaaggacctggcggtgcttcatatccctctagaggagcctgttctgtaatcgataaaccccgatcaacctcaccacctcttgctcagcctatataccgccatcttcagcaaaccctgatgaaggctacaaagtaagcgcaagtacccacgtaaagacgttaggtcaaggtgtagcccatgaggtggcaagaaatgggctacattttctaccccagaaaactacgatagcccttatgaaacttaagggtcgaaggtggatttagcagtaaactgagagtagagtgcttagttgaacagggccctgaagcgcgtacacaccgcccgtcaccctcctcaagtatacttcaaaggacatttaactaaaacccctacgcatttatatagaggagacaagtcgtaacatggtaagtgtactggaaagtgcacttggacgaaccagagtgtagcttaacacaaagcacccaacttacacttaggagatttcaacttaacttgaccgctctgagctaaacctagccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctcaacacccactacctaaaaaatcccaaacatataactgaactcctcacacccaattggaccaatctatcaccctatagaagaactaatgttagtataagtaacatgaaaacattctcctccgcataagcctgcgtcagatcaaaacactgaactgacaattaacagcccaatatctacaatcaaccaacaagtcattattaccctcactgtcaacccaacacaggcatgctcataaggaaaggttaaaaaaagtaaaaggaactcggcaaaccttaccccgcctgtttaccaaaaacatcacctctagcatcaccagtattagaggcaccgcctgcccagtgacacatgtttaacggccgcggtaccctaaccgtgcaaaggtagcataatcacttgttccttaaatagggacctgtatgaatggctccacgagggttcagctgtctcttacttttaaccagtgaaattgacctgcccgtgaagaggcgggcatgacacagcaagacgagaagaccctatggagctttaatttattaatgcaaacagtacctaacaaacccacaggtcctaaactaccaaacctgcattaaaaatttcggttggggcgacctcggagcagaacccaacctccgagcagtacatgctaagacttcaccagtcaaagcgaactactatactcaattgatccaataacttgaccaacggaacaagttaccctagggataacagcgcaatcctattctagagtccatatcaacaatagggtttacgacctcgatgttggatcaggacatcccgatggtgcagccgctattaaaggttcgtttgttcaacgattaaagtcctacgtgatctgagttcagaccggagtaatccaggtcggtttctatctacttcaaattcctccctgtacgaaaggacaagagaaataaggcctacttcacaaagcgccttcccccgtaaatgatatcatctcaacttagtattatacccacacccacccaagaacagggtttgttaagatggcagagcccggtaatcgcataaaacttaaaactttacagtcagaggttcaattcctcttcttaacaacatacccatggccaacctcctactcctcattgtacccattctaatcgcaatggcattcctaatgcttaccgaacgaaaaattctaggctatatacaactacgcaaaggccccaacgttgtaggcccctacgggctactacaacccttcgctgacgccataaaactcttcaccaaagagcccctaaaacccgccacatctaccatcaccctctacatcaccgccccgaccttagctctcaccatcgctcttctactatgaacccccctccccatacccaaccccctggtcaacctcaacctaggcctcctatttattctagccacctctagcctagccgtttactcaatcctctgatcagggtgagcatcaaactcaaactacgccctgatcggcgcactgcgagcagtagcccaaacaatctcatatgaagtcaccctagccatcattctactatcaacattactaataagtggctcctttaacctctccacccttatcacaacacaagaacacctctgattactcctgccatcatgacccttggccataatatgatttatctccacactagcagagaccaaccgaacccccttcgaccttgccgaaggggagtccgaactagtctcaggcttcaacatcgaatacgccgcaggccccttcgccctattcttcatagccgaatacacaaacattattataataaacaccctcaccactacaatcttcctaggaacaacatatgacgcactctcccctgaactctacacaacatattttgtcaccaagaccctacttctaacctccctgttcttatgaattcgaacagcatacccccgattccgctacgaccaactcatacacctcctatgaaaaaacttcctaccactcaccctagcattacttatatgatatgtctccatacccattacaatctccagcattccccctcaaacctaagaaatatgtctgataaaagagttactttgatagagtaaataataggagcttaaacccccttatttctaggactatgagaatcgaacccatccctgagaatccaaaattctccgtgccacctatcacaccccatcctaaagtaaggtcagctaaataagctatcgggcccataccccgaaaatgttggttatacccttcccgtactaattaatcccctggcccaacccgtcatctactctaccatctttgcaggcacactcatcacagcgctaagctcgcactgattttttacctgagtaggcctagaaataaacatgctagcttttattccagttctaaccaaaaaaataaaccctcgttccacagaagctgccatcaagtatttcctcacgcaagcaaccgcatccataatccttctaatagctatcctcttcaacaatatactctccggacaatgaaccataaccaatactaccaatcaatactcatcattaataatcataatggctatagcaataaaactaggaatagccccctttcacttctgagtcccagaggttacccaaggcacccctctgacatccggcctgcttcttctcacatgacaaaaactagcccccatctcaatcatataccaaatctctccctcactaaacgtaagccttctcctcactctctcaatcttatccatcatagcaggcagttgaggtggattaaaccaaacccagctacgcaaaatcttagcatactcctcaattacccacataggatgaataatagcagttctaccgtacaaccctaacataaccattcttaatttaactatttatattatcctaactactaccgcattcctactactcaacttaaactccagcaccacgaccctactactatctcgcacctgaaacaagctaacatgactaacacccttaattccatccaccctcctctccctaggaggcctgcccccgctaaccggctttttgcccaaatgggccattatcgaagaattcacaaaaaacaatagcctcatcatccccaccatcatagccaccatcaccctccttaacctctacttctacctacgcctaatctactccacctcaatcacactactccccatatctaacaacgtaaaaataaaatgacagtttgaacatacaaaacccaccccattcctccccacactcatcgcccttaccacgctactcctacctatctccccttttatactaataatcttatagaaatttaggttaaatacagaccaagagccttcaaagccctcagtaagttgcaatacttaatttctgcaacagctaaggactgcaaaaccccactctgcatcaactgaacgcaaatcagccactttaattaagctaagcccttactagaccaatgggacttaaacccacaaacacttagttaacagctaagcaccctaatcaactggcttcaatctacttctcccgccgccgggaaaaaaggcgggagaagccccggcaggtttgaagctgcttcttcgaatttgcaattcaatatgaaaatcacctcggagctggtaaaaagaggcctaacccctgtctttagatttacagtccaatgcttcactcagccattttacctcacccccactgatgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagctcacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctagacaaaaaaggaaggaatcgaaccccccaaagctggtttcaagccaaccccatggcctccatgactttttcaaaaaggtattagaaaaaccatttcataactttgtcaaagttaaattataggctaaatcctatatatcttaatggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatagcaccccctctaccccctctagagcccactgtaaagctaacttagcattaaccttttaagttaaagattaagagaaccaacacctctttacagtgaaatgccccaactaaatactaccgtatggcccaccataattacccccatactccttacactattcctcatcacccaactaaaaatattaaacacaaactaccacctacctccctcaccaaagcccataaaaataaaaaattataacaaaccctgagaaccaaaatgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgatagccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcgcagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataatgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatggtggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaactaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtcttactcttttagtataaatagtaccgttaacttccaattaactagttttgacaacattcaaaaaagagtaataaacttcgccttaattttaataatcaacaccctcctagccttactactaataattattacattttgactaccacaactcaacggctacatagaaaaatccaccccttacgagtgcggcttcgaccctatatcccccgcccgcgtccctttctccataaaattcttcttagtagctattaccttcttattatttgatctagaaattgccctccttttacccctaccatgagccctacaaacaactaacctgccactaatagttatgtcatccctcttattaatcatcatcctagccctaagtctggcctatgagtgactacaaaaaggattagactgagccgaattggtatatagtttaaacaaaacgaatgatttcgactcattaaattatgataatcatatttaccaaatgcccctcatttacataaatattatactagcatttaccatctcacttctaggaatactagtatatcgctcacacctcatatcctccctactatgcctagaaggaataatactatcgctgttcattatagctactctcataaccctcaacacccactccctcttagccaatattgtgcctattgccatactagtctttgccgcctgcgaagcagcggtgggcctagccctactagtctcaatctccaacacatatggcctagactacgtacataacctaaacctactccaatgctaaaactaatcgtcccaacaattatattactaccactgacatgactttccaaaaagcacataatttgaatcaacacaaccacccacagcctaattattagcatcatccccctactattttttaaccaaatcaacaacaacctatttagctgttccccaaccttttcctccgaccccctaacaacccccctcctaatactaactacctgactcctacccctcacaatcatggcaagccaacgccacttatccagcgaaccactatcacgaaaaaaactctacctctctatactaatctccctacaaatctccttaattataacattcacagccacagaactaatcatattttatatcttcttcgaaaccacacttatccccaccttggctatcatcacccgatgaggcaaccagccagaacgcctgaacgcaggcacatacttcctattctacaccctagtaggctcccttcccctactcatcgcactaatttacactcacaacaccctaggctcactaaacattctactactcactctcactgcccaagaactatcaaactcctgagccaacaacttaatatgactagcttacacaatagcttttatagtaaagatacctctttacggactccacttatgactccctaaagcccatgtcgaagcccccatcgctgggtcaatagtacttgccgcagtactcttaaaactaggcggctatggtataatacgcctcacactcattctcaaccccctgacaaaacacatagcctaccccttccttgtactatccctatgaggcataattataacaagctccatctgcctacgacaaacagacctaaaatcgctcattgcatactcttcaatcagccacatagccctcgtagtaacagccattctcatccaaaccccctgaagcttcaccggcgcagtcattctcataatcgcccacggactcacatcctcattactattctgcctagcaaactcaaactacgaacgcactcacagtcgcatcataatcctctctcaaggacttcaaactctactcccactaatagctttttgatgacttctagcaagcctcgctaacctcgccttaccccccactattaacctactgggagaactctctgtgctagtaaccacgttctcctgatcaaatatcactctcctacttacaggactcaacatactagtcacagccctatactccctctacatatttaccacaacacaatggggctcactcacccaccacattaacaacataaaaccctcattcacacgagaaaacaccctcatgttcatacacctatcccccattctcctcctatccctcaaccccgacatcattaccgggttttcctcttgtaaatatagtttaaccaaaacatcagattgtgaatctgacaacagaggcttacgaccccttatttaccgagaaagctcacaagaactgctaactcatgcccccatgtctaacaacatggctttctcaacttttaaaggataacagctatccattggtcttaggccccaaaaattttggtgcaactccaaataaaagtaataaccatgcacactactataaccaccctaaccctgacttccctaattccccccatccttaccaccctcgttaaccctaacaaaaaaaactcatacccccattatgtaaaatccattgtcgcatccacctttattatcagtctcttccccacaacaatattcatgtgcctagaccaagaagttattatctcgaactgacactgagccacaacccaaacaacccagctctccctaagcttcaaactagactacttctccataatattcatccctgtagcattgttcgttacatggtccatcatagaattctcactgtgatatataaactcagacccaaacattaatcagttcttcaaatatctactcattttcctaattaccatactaatcttagttaccgctaacaacctattccaactgttcatcggctgagagggcgtaggaattatatccttcttgctcatcagttgatgatacgcccgagcagatgccaacacagcagccattcaagcagtcctatacaaccgtatcggcgatatcggtttcatcctcgccttagcatgatttatcctacactccaactcatgagacccacaacaaatagcccttctaaacgctaatccaagcctcaccccactactaggcctcctcctagcagcagcaggcaaatcagcccaattaggtctccacccctgactcccctcagccatagaaggccccaccccagtctcagccctactccactcaagcactatagttgtagcaggaatcttcttactcatccgcttccaccccctagcagaaaatagcccactaatccaaactctaacactatgcttaggcgctatcaccactctgttcgcagcagtctgcgcccttacacaaaatgacatcaaaaaaatcgtagccttctccacttcaagtcaactaggactcataatagttacaatcggcatcaaccaaccacacctagcattcctgcacatctgtacccacgccttcttcaaagccatactatttatgtgctccgggtccatcatccacaaccttaacaatgaacaagatattcgaaaaataggaggactactcaaaaccatacctctcacttcaacctccctcaccattggcagcctagcattagcaggaatacctttcctcacaggtttctactccaaagaccacatcatcgaaaccgcaaacatatcatacacaaacgcctgagccctatctattactctcatcgctacctccctgacaagcgcctatagcactcgaataattcttctcaccctaacaggtcaacctcgcttccccacccttactaacattaacgaaaataaccccaccctactaaaccccattaaacgcctggcagccggaagcctattcgcaggatttctcattactaacaacatttcccccgcatcccccttccaaacaacaatccccctctacctaaaactcacagccctcgctgtcactttcctaggacttctaacagccctagacctcaactacctaaccaacaaacttaaaataaaatccccactatgcacattttatttctccaacatactcggattctaccctagcatcacacaccgcacaatcccctatctaggccttcttacgagccaaaacctgcccctactcctcctagacctaacctgactagaaaagctattacctaaaacaatttcacagcaccaaatctccacctccatcatcacctcaacccaaaaaggcataattaaactttacttcctctctttcttcttcccactcatcctaaccctactcctaatcacataacctattcccccgagcaatctcaattacaatatatacaccaacaaacaatgttcaaccagtaaccactactaatcaacgcccataatcatacaaagcccccgcaccaataggatcctcccgaatcaaccctgacccctctccttcataaattattcagcttcctacactattaaagtttaccacaaccaccaccccatcatactctttcacccacagcaccaatcctacctccatcgctaaccccactaaaacactcaccaagacctcaacccctgacccccatgcctcaggatactcctcaatagccatcgctgtagtatatccaaagacaaccatcattccccctaaataaattaaaaaaactattaaacccatataacctcccccaaaattcagaataataacacacccgaccacaccgctaacaatcagtactaaacccccataaataggagaaggcttagaagaaaaccccacaaaccccattactaaacccacactcaacagaaacaaagcatacatcattattctcgcacggactacaaccacgaccaatgatatgaaaaaccatcgttgtatttcaactacaagaacaccaatgaccccaatacgcaaaattaaccccctaataaaattaattaaccactcattcatcgacctccccaccccatccaacatctccgcatgatgaaacttcggctcactccttggcgcctgcctgatcctccaaatcaccacaggactattcctagccatacactactcaccagacgcctcaaccgccttttcatcaatcgcccacatcactcgagacgtaaattatggctgaatcatccgctaccttcacgccaatggcgcctcaatattctttatctgcctcttcctacacatcgggcgaggcctatattacggatcatttctctactcagaaacctgaaacatcggcattatcctcctgcttgcaactatagcaacagccttcataggctatgtcctcccgtgaggccaaatatcattctgaggggccacagtaattacaaacttactatccgccatcccatacattgggacagacctagttcaatgaatctgaggaggctactcagtagacagtcccaccctcacacgattctttacctttcacttcatcttacccttcattattgcagccctagcagcactccacctcctattcttgcacgaaacgggatcaaacaaccccctaggaatcacctcccattccgataaaatcaccttccacccttactacacaatcaaagacgccctcggcttacttctcttccttctctccttaatgacattaacactattctcaccagacctcctaggcgacccagacaattataccctagccaaccccttaaacacccctccccacatcaagcccgaatgatatttcctattcgcctacacaattctccgatccgtccctaacaaactaggaggcgtccttgccctattactatccatcctcatcctagcaataatccccatcctccatatatccaaacaacaaagcataatatttcgcccactaagccaatcactttattgactcctagccgcagacctcctcattctaacctgaatcggaggacaaccagtaagctacccttttaccatcattggacaagtagcatccgtactatacttcacaacaatcctaatcctaataccaactatctccctaattgaaaacaaaatactcaaatgggcctgtccttgtagtataaactaatacaccagtcttgtaaaccggagacgaaaacctttttccaaggacaaatcagagaaaaagtctttaactccaccattagcacccaaagctaagattctaatttaaactattctctgttctttcatggggaagcagatttgggtaccacccaagtattgactcacccatcaacaaccgctatgtatttcgtacattactgccagccaccatgaatattgtacggtaccataaatacttgaccacctgtagtacataaaaacccaacccacatcaaaccccccccccccatgcttacaagcaagtacagcaatcaaccttcaactatcacacatcaactgcaactccaaagccacccctcacccactaggataccaacaaacctacccacccttaacagtacatagtacataaagtcatttaccgtacatagcacattacagtcaaatcccttctcgtccccatggatgacccccctcagataggggtcccttgaccaccatcctccgtgaaatcaatatcccgcacaagagtgctactctcctcgctccgggcccataacacttgggggtagctaaagtgaactgtatccgacatctggttcctacttcagggccataaagcctaaatagcccacacgttccccttaaataagacatcacgatg